(19) United States
(12) Patent Application Publication
(10) Pub. No.: US 2009/0170845 A1
(43) Pub. Date: Jul. 2, 2009

(54) USE OF IMIDAZO[2,1-b]-1,3,4-THIADIAZOLE-2-SULFONAMIDE COMPOUNDS TO TREAT NEUROPATHIC PAIN

(75) Inventors: Jon Durkin, Montreal (CA); Kimberley Hewitt, Montreal (CA); Peter Winocour, Montreal (CA)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: AEGERA THERAPEUTICS INC., Montreal, Quebec (CA)

(21) Appl. No.: 12/296,810

(22) PCT Filed: Apr. 13, 2007
(86) PCT No.: PCT/CA2007/000627

§ 371 Date: Nov. 24, 2008

(30) Foreign Application Priority Data
Apr. 13, 2006 (US) 60791473
May 11, 2006 (US) 60799480

(51) Int. Cl. A61K 31/535; A61K 31/433

(52) U.S. Cl. 514/233.2; 514/363

(57) ABSTRACT

Disclosed herein are methods and compositions for treating and/or prophylaxis of neuropathic pain in a subject. The methods comprise administering to the subject suffering from neuropathic pain, a therapeutically effective amount of a compound, according to Formula I:

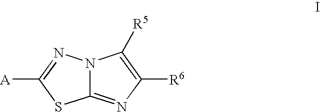

or a salt thereof, wherein A, R⁵ and R⁶ are defined herein.

### FIELD OF THE INVENTION

The present invention concerns the use of imidazo[2,1-b]-1,3,4-thiadiazole-2-sulfonamide compounds as pharmaceutical agents to treat neuropathic pain in mammals, particularly humans.

### BACKGROUND OF THE INVENTION

Neuropathic pain is the result of an injury or malfunction in the peripheral or central nervous system. Neuropathic pain conditions are characterized by hyperesthesia (enhanced sensitivity to natural stimuli), hyperalgesia (abnormal sensitivity to pain), allodynia (pain from stimuli which are not normally painful) and/or spontaneous burning pain. In humans, neuropathic pains tend to be chronic. The pain is often triggered by an injury, but this injury may or may not involve actual damage to the nervous system. Nerves can be infiltrated or compressed by tumors, strangulated by scar tissue, or inflamed by infection or hosting a viral infection such as Herpes virus or Human Immunodeficiency virus. The pain frequently has burning, lacerating, or electric shock qualities. Persistent allodynia, pain resulting from a non-painful stimulus such as a light touch, is also a common characteristic of neuropathic pain. The pain may persist for months or years beyond the apparent healing of any damaged tissues. In this setting, pain signals no longer represent an alarm about ongoing or impending injury, instead the alarm system itself is malfunctioning. Examples include post herpetic (or post-shingles) neuralgia, reflex sympathetic dystrophy/causalgia (nerve trauma), components of cancer pain, phantom limb pain, entrapment neuropathy (e.g., carpal tunnel syndrome), and peripheral polyneuropathy (widespread nerve damage). Among the many causes of neuropathic pain, diabetes is the most common, but the condition can also be caused by chronic alcohol use, exposure to other toxins (including many chemotherapies), vitamin deficiencies, and a large variety of other medical conditions—it is not unusual for the cause of the condition to go undiagnosed.

Neuropathic pain has traditionally been treated using narcotic analgesics such as opioids. Administration of various opioid derivatives such as morphine may provide some degree of relief but at doses that are impractical for lifelong treatments (Bennett, Hosp. Practice Vol. 33, pages 95 to 114, 1998). Pregabalin has recently been approved for the treatment of neuropathic pain associated with diabetic peripheral neuropathy (DN) and postherpetic neuralgia, however, it demonstrates limited clinical efficacy and requires multiple daily dosing. Other pharmaceutical agents used to treat neuropathic pain include anti-depressants, anti-convulsants, and local anesthetics. Although many of these agents provide symptomatic relief of pain, their long term use is complicated by limited clinical efficacy, short duration of action and un-related modes of action; with characteristic side effects such as dizziness, somnolence, ataxia, confusion, abnormal thinking, blurred vision, incoordination, and the development of dependence or addiction. As a whole, these classes of agents have met with limited clinical success, necessitating the need to develop alternate therapies for the treatment, prophylaxis or cure for neuropathic pain.

We previously disclosed that a family of imidazo[2,1-b]-1,3,4-thiadiazole-2-sulfonamides demonstrated in vitro neuroprotective effects, characterized by protection of Superior Cervical Ganglion (SCG) neurons subjected to NGF withdrawal, from apoptotic death. These compounds also protect cultured neurons from multiple neurotoxic insults including treatment with cytotoxic agents such as taxanes, platinum derivatives and vinca alkaloids. A selection of these compounds, and their N-acyl prodrug derivatives, demonstrated efficacy in animal models of peripheral neuropathy, resulting in enhanced functional recovery from noxious peripheral stimuli, such as those causing chemotherapy-induced neuropathy (CTIN) Functional recovery was measured in terms of recovered nerve conduction velocity and improved gait mobility. The compounds showed enhanced axonal re-growth in a nerve damage model and improved electroretinograph function following retinal ischemia. Due to their properties of protection of cultured neurons from neurotoxic insults such as Neuronal Growth Factor (NGF) withdrawal, it was believed that these compounds acted on the neurotrophin survival signaling pathway. NGF replacement therapy has been demonstrated as a clinically relevant treatment for diabetic peripheral neuropathy and HIV-induced peripheral neuropathy, however, it was shown to be associated with an unacceptable level of induced hyperalgesia and injection site local pain. Clearly, it would be useful to identify compounds which attempt to treat an underlying neuropathy without inducing or exacerbating a state of neuropathic pain.

This invention relates to the unexpected finding that compounds of the present invention are capable of treating neuropathic painful states such as those induced by diabetes, and inflammatory mediators, which result in rapid onset, long lasting pain relief. Further, compounds of this class appear to prevent or reverse nerve damage in a model of Diabetic Neuropathy, as indicated by assessment of both motor and sensory nerve conduction velocity (NCV) measurements and reversal of loss of axonal diameter and morphology.

Mechanism of action studies have recently demonstrated that a common molecular link in many peripheral neurotoxic insults is the induction of JNK phosphorylation in neurons, for example dorsal horn neurons in cell culture, which results in induction of the neuronal apoptotic state. Compounds of the present invention are capable of blocking this induction of JNK phosphorylation in neuronal cell culture in vitro.

A growing body of recent literature demonstrates that upregulated JNK phosphorylation and activity is also observed in-vivo in neurons of the PNS in preclinical models of diabetic neuropathy (DN) and in models of neuropathic pain (Daulhac et al., 2006; Zhuang et al., 2006; Middlemas, Agthong, & Tomlinson, 2006). Similarly, nerve cell JNK phosphorylation has been recently been observed in models of inflammatory pain (Doya et al., 2005; Liu et al., 2007). Spinal application of a JNK inhibitor was shown to be effective at reversing pain states in animals (Zhuang et al., 2006; Liu et al., 2007). Aberrant JNK phosphorylation has also been observed in nerve biopsy samples from diabetic patients (Purves et al., 2001). This mechanistic link supports our observations of neuropathic pain relief in disease models, and furthermore predicts that compounds of the class disclosed herein, will find use in the treatment of multiple states of neuropathic pain in the human condition.

### SUMMARY OF THE INVENTION

The present invention provides compositions and methods for treating the aforesaid types of neuropathic pain. The compositions and methods employ acylated and non-acylated imidazo[2,1-b]-1,3,4-thiadiazole-2-sulfonamide compounds as their active agents. Many of the compounds have already been disclosed in commonly-owned U.S. patent application Ser. No. 10/498,548 and published PCT application PCT CA02/01942 and U.S. patent application Ser. No. 10/599,675, published PCT application PCT/CA2004/000873.

The imidazo[2,1-b]-1,3,4-thiadiazole-2-sulfonamides of the instant invention display unexpected onset and duration of action in several in vivo models of diabetic neuropathic and inflammatory neuropathic pain when administered by systemic routes of administration. Further, a subset of these compounds demonstrate efficacy when given orally, the preferred route for chronic treatment.

Unexpectedly, these compounds do not behave like typical analgesics such as NSAIDS, opioids or gabapentin which are only active for 2-6 hours after a single administration. The pain relief provided by compounds of the instant invention was shown to last for up to 24 hrs after a single dose of compound.

Further, compounds of this class arrear to prevent or reverse nerve damage in a model of DN, as indicated by assessment of both motor and sensory nerve conduction velocity (NCV) measurements and axonal morphology.

According to an embodiment of the present invention, there is provided a method of treating and/or prophylaxis of neuropathic pain, comprising: administering to a subject suffering from neuropathic pain, a therapeutically effective amount of one or more acylated or non-acylated imidazo[2,1-b]-1,3,4-thiadiazole-2-sulfonamide compounds.

According to another embodiment of the present invention, there is provided a method of treating and/or prophylaxis of neuropathic pain, comprising: administering to a subject suffering from neuropathic pain, a therapeutically effective amount of a compound, according to Formula I:

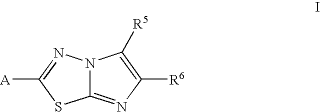

or a salt thereof,
wherein:
n is 1 or 2;
m is an integer from 0 to 22;
s is an integer from 0 to 6;
p is an integer from 0 to 1;

### Y is NH, O or S;

### A is —S(O)₂NR¹R²;

R¹ and R² are independently selected from:
1) H,
2) C₁-C₆ alkyl, or
3) C(O)R⁴;

### R⁴ is

1) C₁-C₁₈ alkyl,
2) aryl,
3) heteroaryl,
4) (CH₂)ₛ—(C(O))ₚ—(OCH₂CH₂)ₘOR¹⁰; or
5) C₁-C₆ alkyl-NR¹¹R¹²,
   wherein alkyl is optionally substituted with one or more R¹⁵ substituents; and aryl and heteroaryl are optionally substituted with one or more R²⁰ substituents

### R⁵ is:

1) H,
2) halogen,
3) C₁-C₆ alkyl,
4) phenyl,
5) S-aryl, or
6) S-heteroaryl,
   wherein the aryl and the heteroaryl are optionally substituted with one or more R²⁰ substituents;

### R⁶ is

1) haloalkyl,
2) adamantyl,
3) aryl,
4) heteroaryl,
5) fused phenyl-cycloalkyl substituted with alkyl, or
6) fused phenyl-heterocyclyl optionally substituted with cycloalkyl,
   wherein the aryl and the heteroaryl are optionally substituted with one or more substituents independently selected from R²⁰;

### R¹⁰ is

1) C₁-C₆ alkyl,
2) C₃-C₇ cycloalkyl,
3) haloalkyl,
4) C₂-C₆ alkenyl;
5) C₂-C₆ alkynyl;
6) C₅-C₇ cycloalkenyl,
7) aryl,
8) heteroaryl, or
9) heterocyclyl,
   wherein the alkyl, cycloalkyl, alkenyl, alkynyl, cycloalkenyl are optionally substituted with one or more R¹⁵ substituents, and the aryl, heteroaryl, heterocyclyl, and biphenyl are optionally substituted with one or more R²⁰ substituents;
   R¹¹ and R¹² are independently selected from:
1) C₁-C₆ alkyl,
2) C₃-C₇ cycloalkyl,
3) haloalkyl,
4) aryl,
5) heteroaryl,
6) heterocyclyl,
7) CO—C₁-C₆ alkyl
8) CO—C₃-C₇ cycloalkyl
9) CO-aryl,
10) CO-heteroaryl,
11) CO-heterocyclyl,
12) C(O)Y—C₁-C₆ alkyl
13) C(O)Y—C₃-C₇ cycloalkyl
14) C(O)Y-aryl,
15) C(O)Y-heteroaryl,
16) C(O)Y-heterocyclyl,
   wherein the alkyl and the cycloalkyl are optionally substituted with one or more R¹⁵ substituents, and the aryl, heteroaryl, heterocyclyl, and biphenyl are optionally substituted with one or more R²⁰ substituents;
   or R¹¹ and R¹² together with the nitrogen atom to which they are bonded form a five, six or seven membered heterocyclic ring optionally substituted with one or more R²⁰ substituents;

### R¹⁵ is

1) NO₂,
2) CN,
3) halogen,
4) C₁-C₆ alkyl,
5) C₃-C₇ cycloalkyl,
6) haloalkyl,
7) aryl,
8) heteroaryl,
9) heterocyclyl,
10) OR¹⁰,
11) S(O)ₙR¹⁰,
12) NR¹¹R¹²,
13) COR¹⁰,
14) CO₂R¹⁴,
15)CONR¹¹R¹², or
16) S(O)ₙNR¹¹R¹²,
   wherein the aryl and heteroaryl are optionally substituted with one or more R¹⁰ substituents;

### R²⁰ is

1) NO₂,
2) CN,
3) N₃,
4) B(OH)₂,
5) adamantyl,
6) halogen,
7) C₁-C₆ alkyl,
8) C₃-C₇ cycloalkyl,
9) aryl,
10) heteroaryl,
11) heterocyclyl,
12) fused phenyl heterocyclyl,
13) haloalkyl,
14) OR¹⁰,
15) SR¹⁰,
16) S(O)ₙR¹⁰,
17) NR¹¹R¹²,
18) COR¹⁰,
   wherein the alkyl, the aryl, the heteroaryl, the heterocyclyl, and the cycloalkyl are optionally substituted with one or more R¹⁵ substituents.

According to another embodiment of the present invention, there is provided a pharmaceutical composition for treating and/or prophylaxis of neuropathic pain, comprising: a pharmaceutically acceptable carrier and a therapeutically effective amount of a compound, according to Formula I:

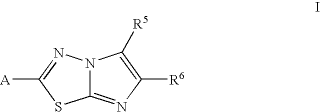

or a salt thereof; wherein A, R⁵ and R⁶ are as defined above.

Accordingly in another embodiment, there is provided a method of treating and/or prophylaxis of neuropathic pain, comprising: administering to a subject suffering from neuropathic pain, in combination, a compound of Formula I, and another agent, in a therapeutically effective amount sufficient to cause reduction of the pain.

Accordingly in another embodiment, there is provided a method of treating and/or prophylaxis of neuropathic pain, comprising: administering to a subject suffering from neuropathic pain, in combination, a composition as described above, and another agent, in a therapeutically effective amount sufficient to cause reduction of the pain.

According to another embodiment of the present invention, there is provided use of a compound of Formula I, or a pharmaceutical composition, as described above, for the treatment and/or prophylaxis of neuropathic pain in a subject.

According to another embodiment of the present invention, there is provided use of a compound of Formula I, or a pharmaceutical composition, as described above in the manufacture of a medicament for the treatment and/or prophylaxis of neuropathic pain in a subject

According to another embodiment of the present invention, there is provided use of a combination of a compound of Formula I or a pharmaceutical composition, as described above, and another agent, for the treatment and/or prophylaxis of neuropathic pain in a subject.

Accordingly in another embodiment, there is provided use of, in combination, a compound of Formula I or a pharmaceutical composition as described above, and another agent, for the manufacture of a medicament for the treatment and/or prophylaxis of neuropathic pain.

### BRIEF DESCRIPTION OF THE DRAWINGS

Further aspects and advantages of the present invention will become better understood with reference to the description in association with the following Figures, wherein:

FIG. 1 is a graph illustrating the impact of compound 150 on sensory nerve conduction velocity (SNCV) in diabetic rats after two months of treatment, with therapy initiated after conduction velocity deficits were already apparent;

FIG. 2 is a graph illustrating the impact of compound 150 on motor nerve conduction velocity (MNCV) in diabetic rats after two months of treatment, with therapy initiated after conduction velocity deficits were already apparent;

FIG. 3 is a graph illustrating a morphometric analysis of sural nerve myelinated axons. Note that D refers to vehicle treated animals, B to compound 150 treated animals, DI indicates diabetic rats, and C indicates nondiabetic age-matched controls; FIG. 3a illustrates mean axon area; FIG. 3b illustrates frequency histogram by size;

FIG. 4 is a graph illustrating a morphometric analysis of sural nerve myelinated axons of larger caliber (greater than 9 microns square). FIG. 4A: mean axon area and FIG. 4B: frequency histogram sorted by size. Note that D refers to vehicle treated animals, B to compound 150 treated animals, DI indicates diabetic rats, and C indicates nondiabetic age-matched controls;

FIG. 5 is a graph illustrating the effect of Compound 150 on Tactile Allodynia in Diabetic rats after 1, 5 and 10 treatments;

FIG. 6 is a graph illustrating the effect of Compound 157 on Tactile Allodynia in Diabetic rats prior to treatment, and after 1, 13 and 14 daily treatments;

FIG. 7 is a graph illustrating the effect of Compound 158 on Tactile Allodynia in Diabetic rats prior to treatment, and after 1, 13 and 14 daily treatments;

FIG. 8 is a graph illustrating the effect of compound 155 on tactile allodynia in diabetic rats 6 hours after a single subcutaneous administration;

FIG. 9 is a graph illustrating the effect of compound 157 on tactile allodynia in diabetic rats 6 hours after subcutaneous administration;

FIG. 10 is a graph illustrating the effect of compound 157 on tactile allodynia in diabetic rats 6 hours after oral administration;

FIG. 11 is a graph illustrating the effect of compound 154 on tactile allodynia in diabetic rats 6 hours after subcutaneous administration;

FIG. 12 is a graph illustrating the effect of compound 158 on tactile allodynia in diabetic rats 6 hours after subcutaneous administration;

FIG. 13 illustrates the effect of compound 160 on tactile allodynia in diabetic rats 6 hours after subcutaneous administration;

FIG. 14 is a graph illustrating the effect of compound 157 on tactile allodynia in diabetic rats 6 hours after the 5^{th} oral administration of drug, given orally once daily over five consecutive days;

FIG. 15 is a graph illustrating the effect of compound 158 on tactile allodynia in diabetic rats 6 hours after the 5^{th} oral administration of drug, given orally once daily over five consecutive days;

FIG. 16 is a graph illustrating the effect of compound 150 on tactile hyperalgesia in the CFA pain model after subcutaneous administration;

FIG. 17 is a graph illustrating the effect of Compound 155 on tactile hyperalgesia in the CFA pain model after subcutaneous administration;

FIG. 18 is a graph illustrating the effect of Compound 157 on tactile hyperalgesia in the CFA pain model after subcutaneous administration;

FIG. 19 is a graph illustrating the effect of Compound 158 on tactile hyperalgesia in the CFA pain model after subcutaneous administration;

FIG. 20 is a graph illustrating the effect of Compound 157 on tactile hyperalgesia in the CFA pain model after oral administration; and

FIG. 21 is a graph illustrating the effect of Compound 157 on tactile hyperalgesia 6 hours after the 5^{th} oral administration of drug, given orally once daily over five consecutive days.

### DETAILED DESCRIPTION OF THE INVENTION

### Definitions

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, C₁-C₆ as in C₁-C₆— alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement. Examples of C₁-C₆-alkyl and C₁-C₄ alkyl as defined above include, but are not limited to, methyl, ethyl, n-propyl, i-propyl, n-butyl, t-butyl, i-butyl, pentyl and hexyl. Also included in this definition is C₁₋₁₈ as in C₁₋₁₈ alkyl, which is defined as including groups having, 1, 2, 3, 4, 5, 6, 7, 8, 9, 10, 11, 12, 13, 14, 15, 16, 17, or 18 carbon atoms in a linear or branched arrangement.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean a monocyclic saturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl” is intended to mean any stable monocyclic or bicyclic aromatic carbon ring containing 6 or 10 carbon atoms. Examples of such aryl substituents include, but are not limited to, phenyl and naphthyl.

As used herein, the term “biphenyl” is intended to mean two phenyl groups bonded together at any one of the available sites on the phenyl ring. For example:

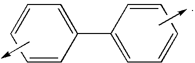

As used herein, the term “fused aryl-C₃-C₇cycloalkyl” is intended to mean an aryl group, as defined herein, which is fused with a cycloalkyl group, as defined herein. The fused aryl-C₃-C₇ cycloalkyl may be connected to another group either at a suitable position on the cycloalkyl ring or the aromatic ring. For example:

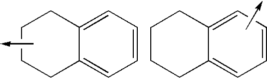

Arrowed lines drawn from the ring system indicate that the bond may be attached to any of the suitable ring atoms.

As used herein, the term “fused heteroaryl-C₃-C₇ cycloalkyl” is intended to mean a heteroaryl group, as defined herein, which is fused with a cycloalkyl group, as defined herein. The fused heteroaryl-C₃-C₇ cycloalkyl may be connected to another group either at a suitable position on the cycloalkyl ring or the heteroaromatic ring.

As used herein, the term “fused aryl-heterocyclyl” is intended to mean a heterocyclyl group, as defined herein, which is fused with an aryl group, as defined herein. The fused aryl-heterocyclyl may be connected to another group either at a suitable position on the aryl ring or the heterocyclyl ring. Examples of fused aryl-heterocyclyls include, but are not limited to benzo[d][1,3]dioxole, 2,3-dihydrobenzo[b][1,4]dioxine and 3,4-dihydro-2H-benzo[b][1,4]dioxepine.

As used herein, the term “fused heteroaryl-heterocyclyl” is intended to mean a heteroaryl group, as defined herein, which is fused with a heterocyclyl group, as defined herein. The fused heteroaryl-heterocyclyl may be connected to another group either at a suitable position on the heteroaryl ring or the heterocyclyl ring.

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, and isoindolinyl,

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, and pyrazolinyl,

As used herein the term “neuropathic pain” is intended to mean pain caused by peripheral nerve trauma, entrapment neuropathy, nerve transaction, including surgery, causaglia, amputation and stump pain, neuroma, and post-choracotomy pain, mononeuropathies such as diabetic, malignant nerve/plexus invasion, ischemic irradiation, connective tissue disease, rheumatoid arthritis, systemic lupus erythematosus, polyarteritis nodosa; polyneuropathies such as diabetic, alcoholic, nutritional, amyloid, Fabry disease, chemical (e.g., chemotherapeutic agents), idiopathic and AIDS neuropathy; root and dorsal root ganglion, prolapsed disk/compression, postherpetic or trigeminal neuralgia, arachnoiditis, root avulsion, tumor compression and surgical rhizotomy; by spinal cord injury such as trauma, transaction, hemisection, Lissauer tract section, syrinx, multiple sclerosis, tumor compression, arteriovenous malformation, Dyscraphism, Vitamin B12 deficiency, hematomyelia, syphilitic myelitis, and Commissural myelotomy; brain stem injury such as Wallenberg's syndrome, multiple sclerosis, tuberculoma, tumor, and syrinx; thalamus injury, such as infarction, tumor, surgical lesions in main, sensory nucleus, and hemorrahage; corrical/subcorrical injury, such as infarction, trauma, tumor, and arteriovenous malformation; as defined in Pain Management by Rochelle Wagner and Robert R. Myers. Other types of painful diabetic peripheral neuropathy, post-herpetic neuralgia, trigeminal neuralgia, post-stroke pain, multiple sclerosis-associated pain, neuropathies-associated pain such as in idiopathic or post-traumatic neuropathy and mononeuritis, HIV-associated neuropathic pain, cancer-associated neuropathic pain, carpal tunnel-associated neuropathic pain, spinal cord injury-associated pain, complex regional pain syndrome, fibromyalgia-associated neuropathic pain, lumbar and cervical pain, reflex sympathic dystrophy, phantom limb syndrome and other chronic and debilitating condition-associated pain syndromes.

As used herein, the term “heteroatom” is intended to mean O, S or N.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

As used herein, the term “therapeutically effective amount” is intended to mean the amount of a compound of the present invention effective to reduce or eliminate the neuropathic pain by treatment and/or prophylaxis.

As used herein, the term “subject” is intended to mean humans and non-human mammals such as primates, cats, dogs, swine, cattle, sheep, goats, horses, rabbits, rats, mice and the like.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The compounds of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

### I. Compounds

Compounds of the present invention may be represented by Formula I. Compounds of the present invention can be synthesized using the chemistry or adaptations thereof, which are disclosed in WO 03/051,890 A1; and WO 2004/111,061 A, the contents of which are hereby incorporated by reference I their entirety.

One subset of compounds of Formula I include compounds of Formula 1a:

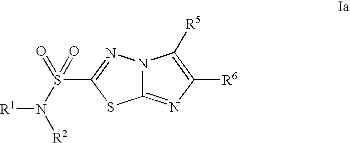

or a salt thereof, wherein R¹, R², R⁵ and R⁶ are as defined hereinabove.

In one subset of Formula 1a, R¹ and R² are individually selected from the group consisting of H, methyl, ethyl, propyl, and butyl. In one example, R¹ and R² are both H.

In one alternative subset of Formula 1a, R² is H and R¹ is C(O)R⁴, wherein R⁴ is described hereinabove.

In one subset of Formula 1a, R⁵ is H, C₁-C₆ alkyl or phenyl. In one example R⁵ is H.

In one subset of Formula 1a, R⁶ is
1) haloalkyl,
2) adamantyl,
3) aryl,
4) heteroaryl,
5) fused phenyl-cycloalkyl substituted with alkyl, or
6) fused phenyl-heterocyclyl optionally substituted with cycloalkyl,
   wherein the aryl and the heteroaryl are optionally substituted with one or more substituents independently selected from R²⁰.

In one subset of the R⁶ described immediately above, R⁶ is phenyl optionally substituted with one or more R²⁰ substituents. In one example, R⁶ is selected from the group consisting of:

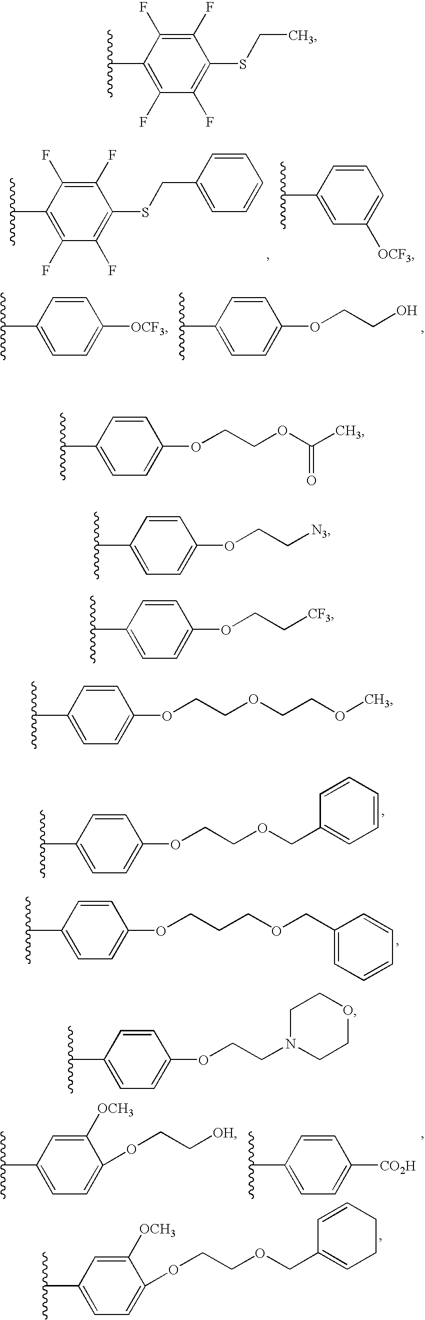

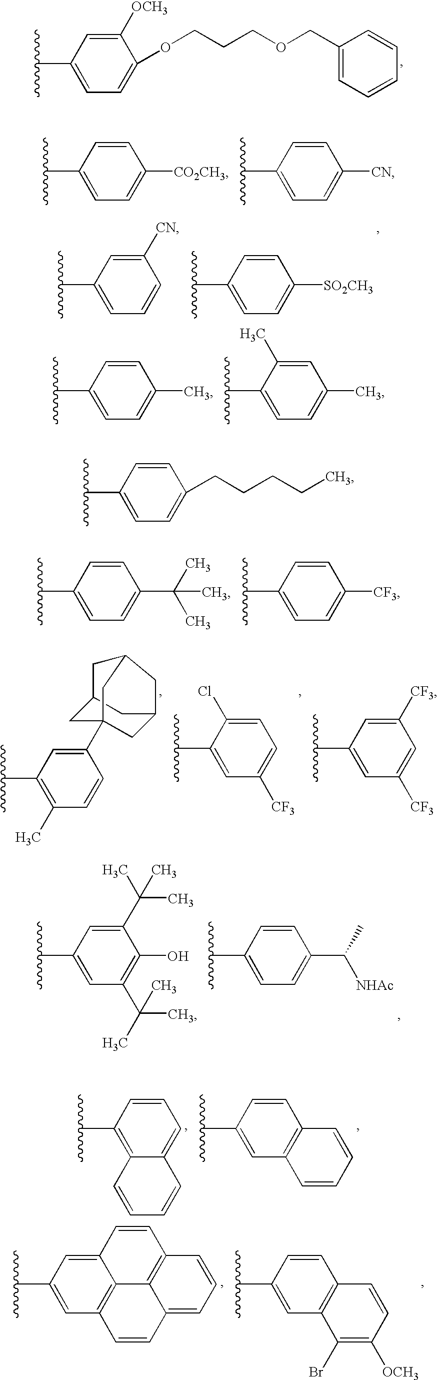

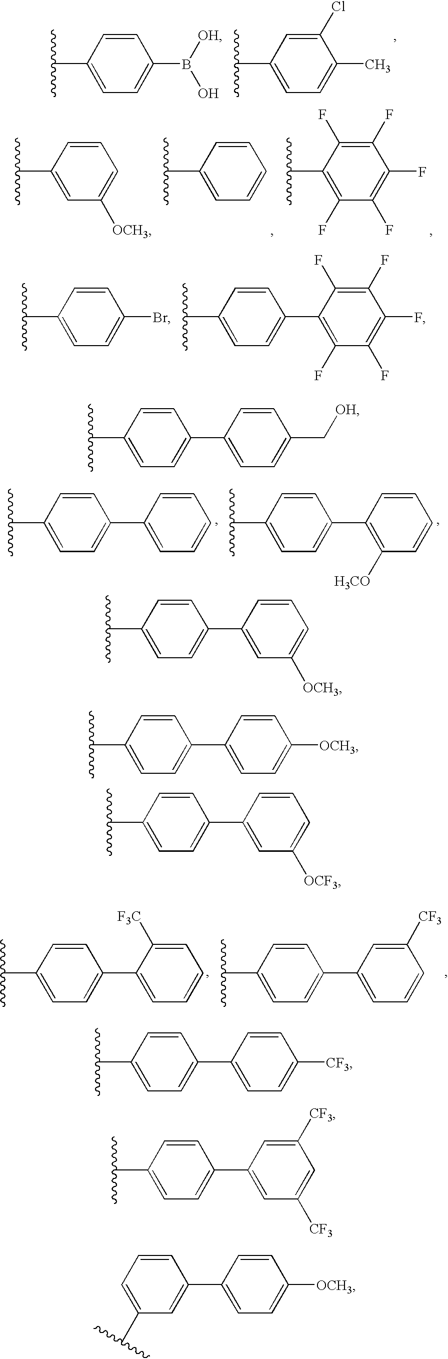

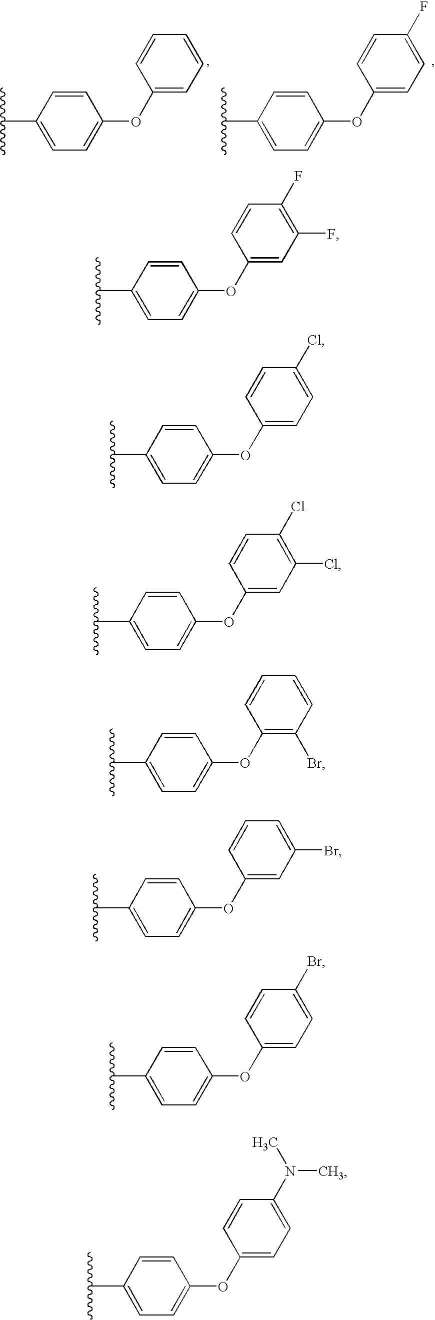

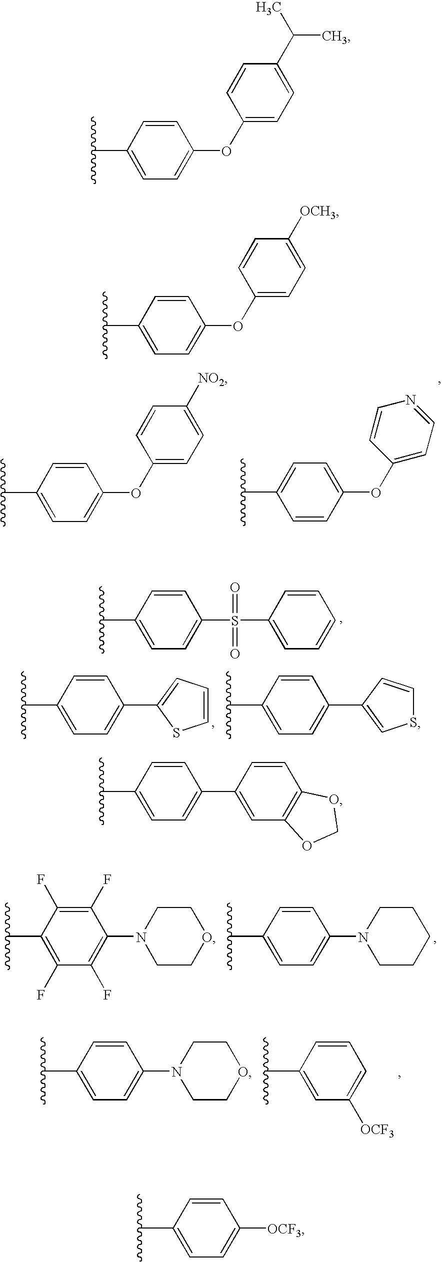

In an alternative subset of Formula 1a, R⁶ is heteroaryl, fused phenyl-cycloalkyl substituted with two or more methyl groups, or fused phenyl-heterocycyl substituted with cyclohexane. In one example, R⁶ is selected from the group consisting of:

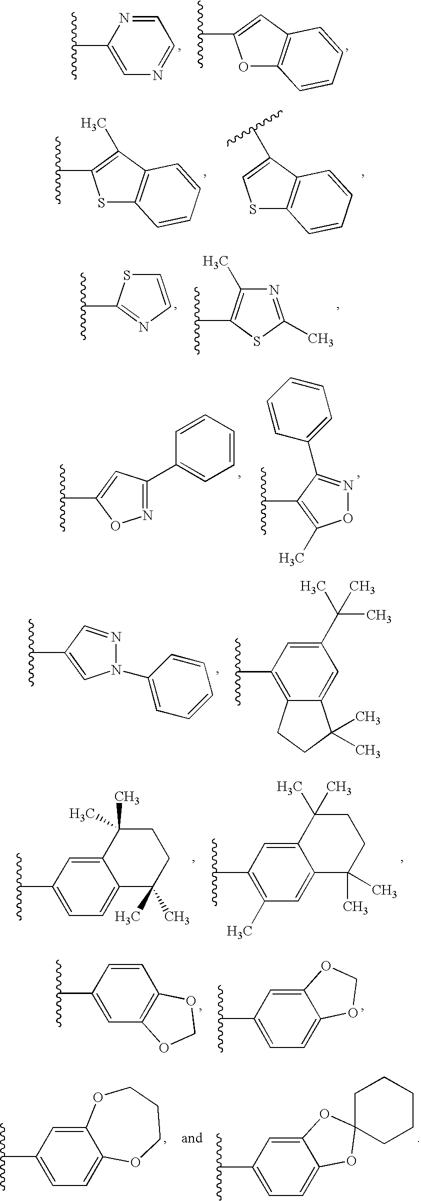

Specific examples of compounds of Formula 1a include:

| No. | Structure |
|---|---|
| 1 | 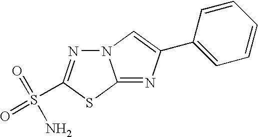 |
| 2 | 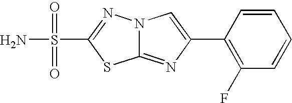 |
| 3 | 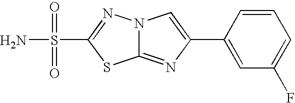 |
| 4 | 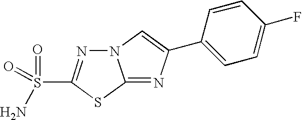 |
| 5 | 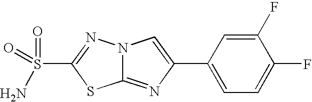 |
| 6 | 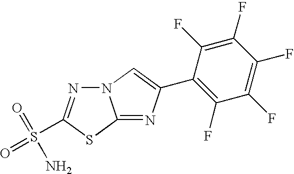 |
| 7 | 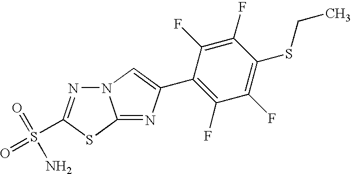 |
| 8 | 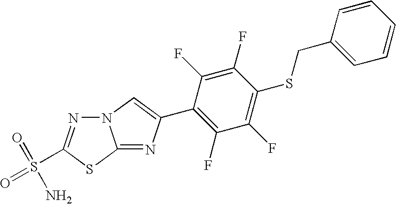 |
| 9 | 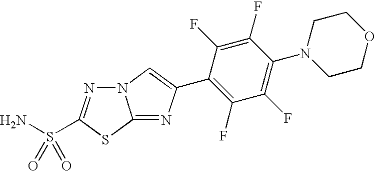 |
| 10 | 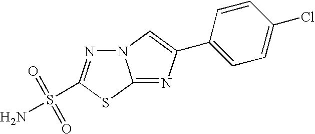 |
| 11 | 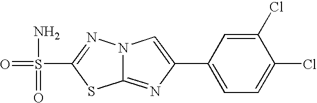 |
| 12 | 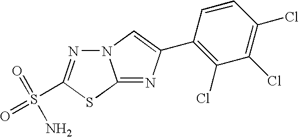 |
| 13 | 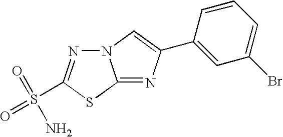 |
| 14 | 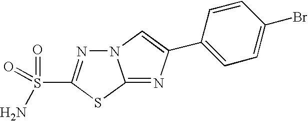 |
| 15 | 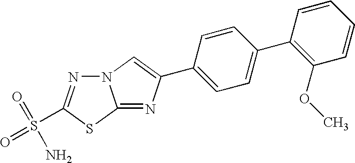 |
| 16 | 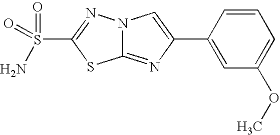 |
| 17 | 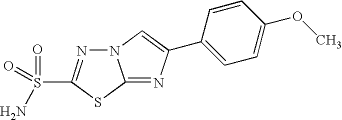 |
| 18 | 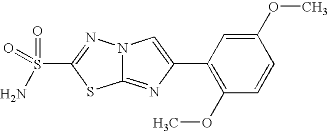 |
| 19 | 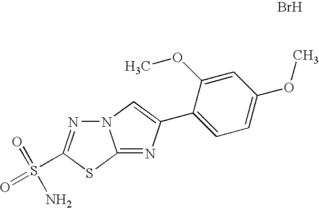 |
| 20 | 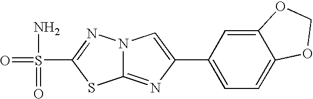 |
| 21 | 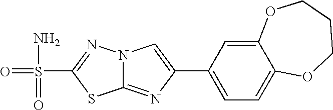 |
| 23 | 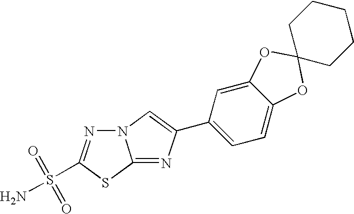 |
| 24 | 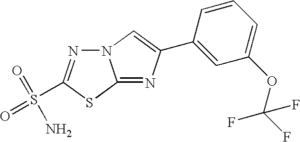 |
| 25 | 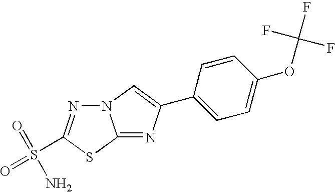 |
| 26 | 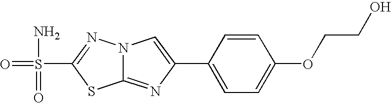 |
| 27 | 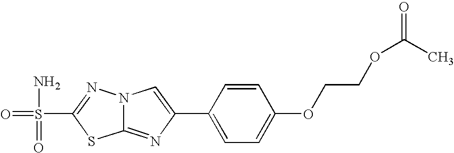 |
| 29 | 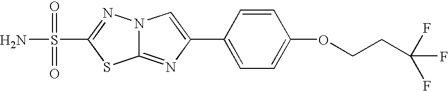 |
| 30 | 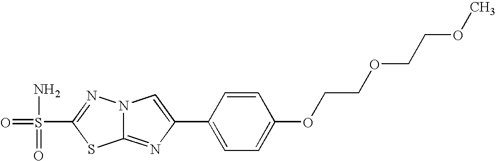 |
| 31 | 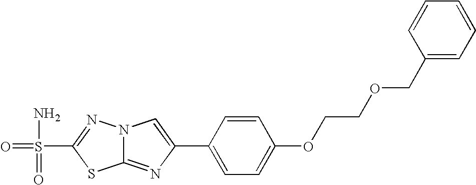 |
| 32 | 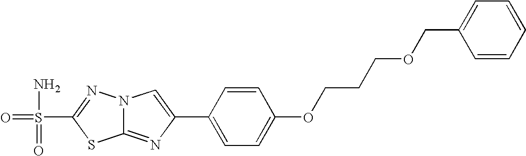 |
| 33 | 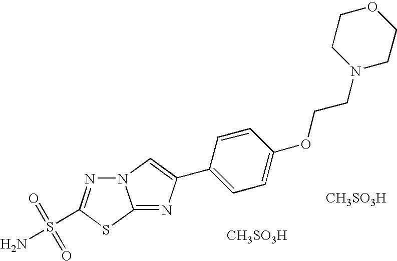 |
| 34 | 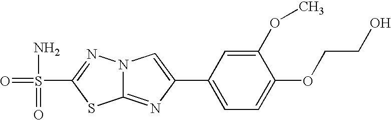 |
| 35 | 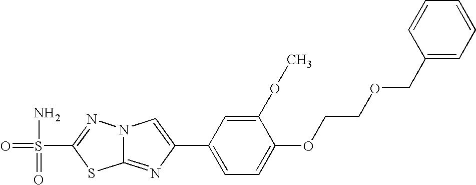 |
| 36 | 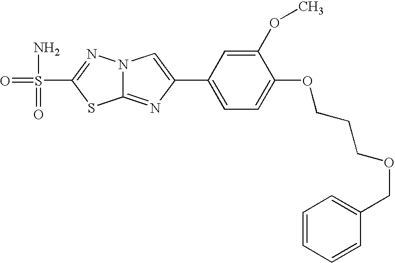 |
| 37 | 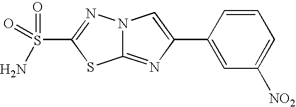 |
| 38 | 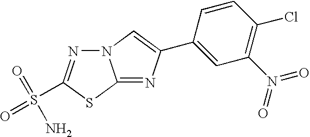 |
| 41 | 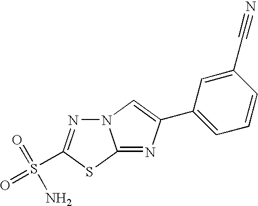 |
| 42 | 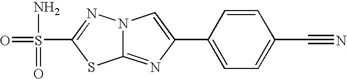 |
| 43 | 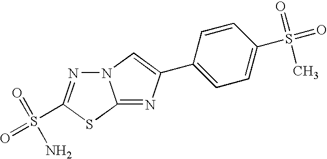 |
| 44 | 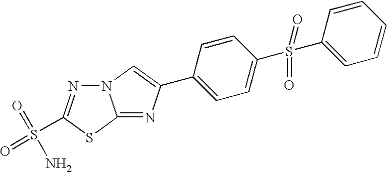 |
| 46 | 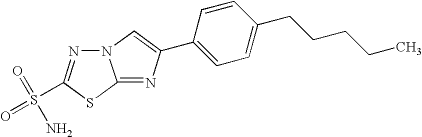 |
| 47 | 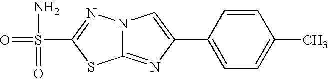 |
| 48 | 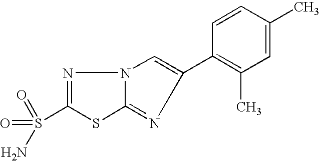 |
| 49 | 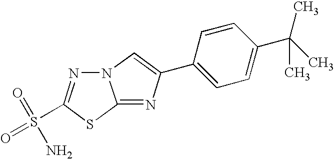 |
| 50 | 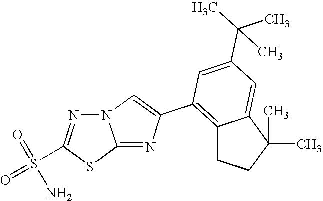 |
| 51 | 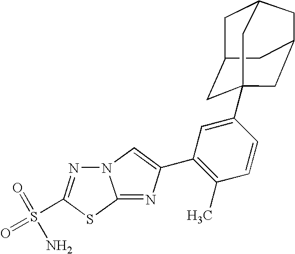 |
| 52 | 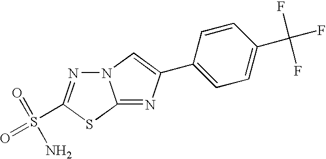 |
| 53 | 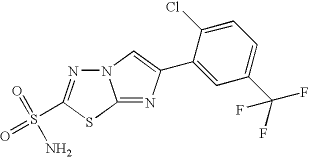 |
| 54 | 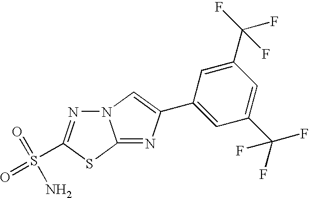 |
| 55 | 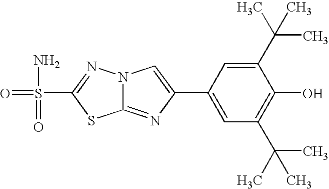 |
| 56 | 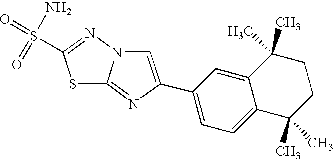 |
| 57 | 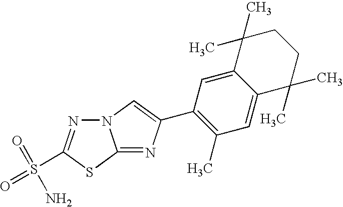 |
| 58 | 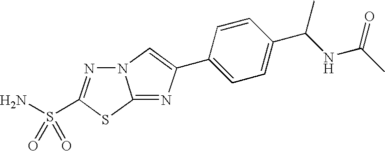 |
| 59 | 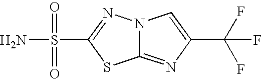 |
| 60 | 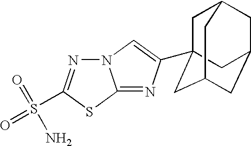 |
| 61 | 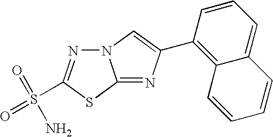 |
| 62 | 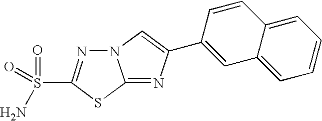 |
| 63 | 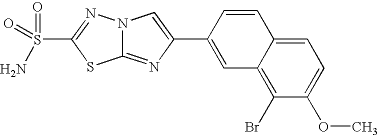 |
| 64 | 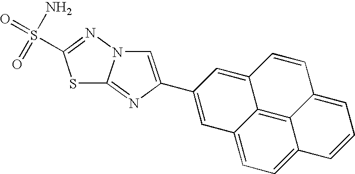 |
| 65 | 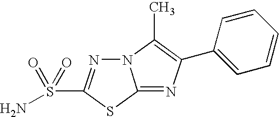 |
| 66 | 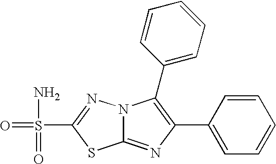 |
| 67 | 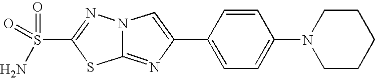 |
| 68 | 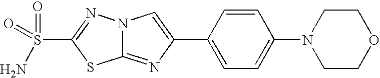 |
| 71 | 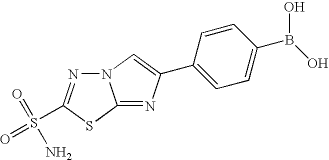 |
| 72 | 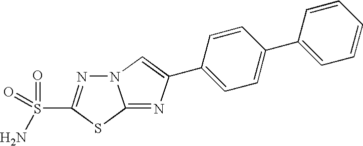 |
| 73 | 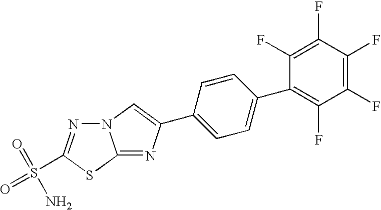 |
| 74 | 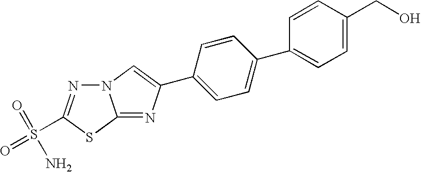 |
| 75 | 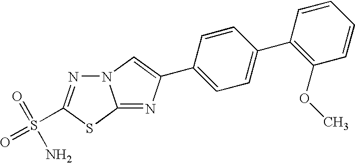 |
| 76 | 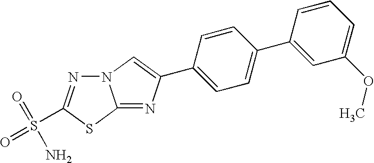 |
| 77 | 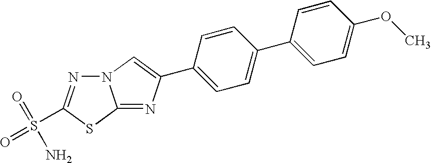 |
| 78 | 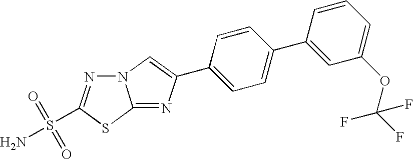 |
| 79 | 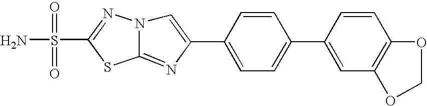 |
| 80 | 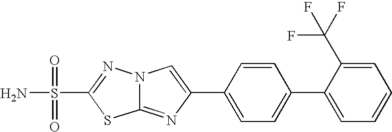 |
| 81 | 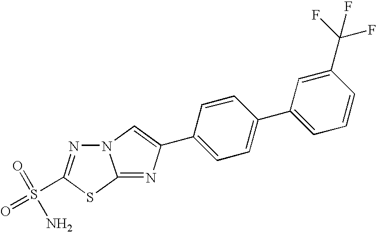 |
| 82 | 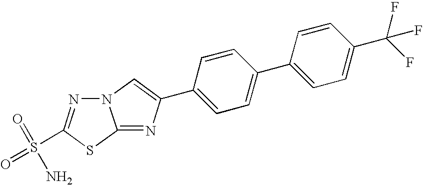 |
| 83 | 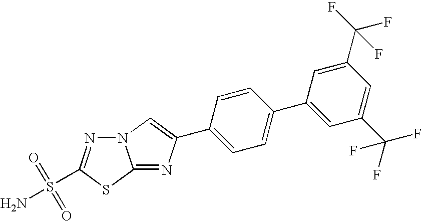 |
| 84 | 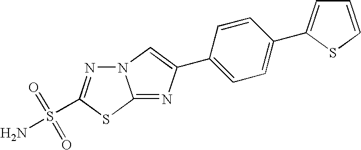 |
| 85 | 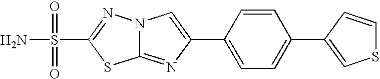 |
| 86 | 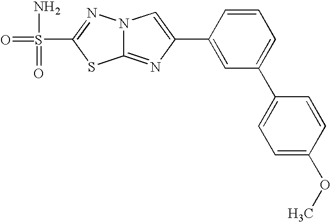 |
| 87 | 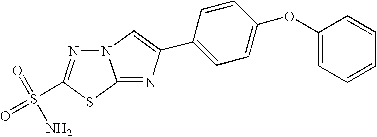 |
| 88 | 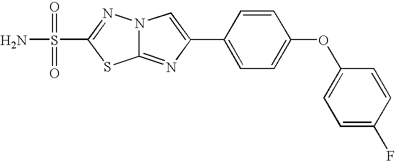 |
| 89 | 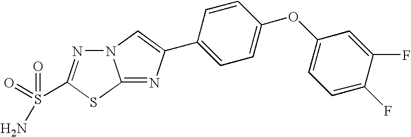 |
| 90 | 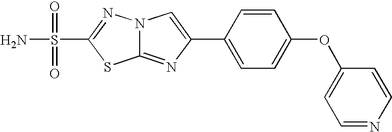 |
| 91 | 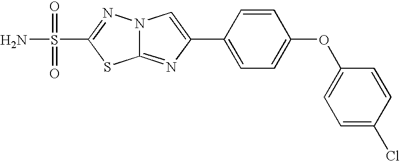 |
| 92 | 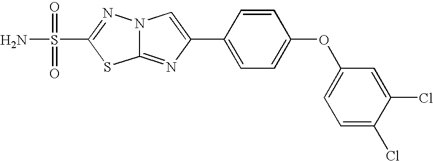 |
| 93 | 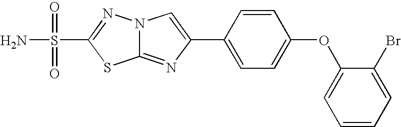 |
| 94 | 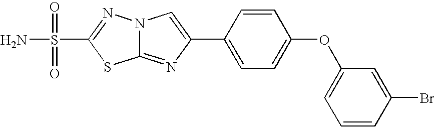 |
| 95 | 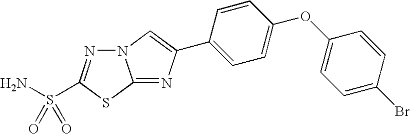 |
| 96 | 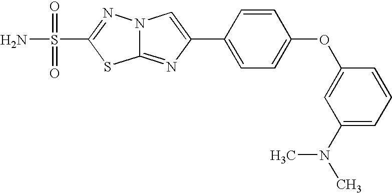 |
| 97 | 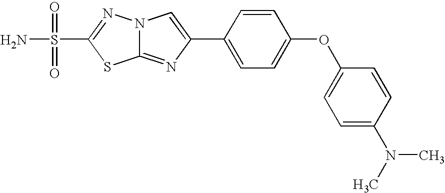 |
| 98 | 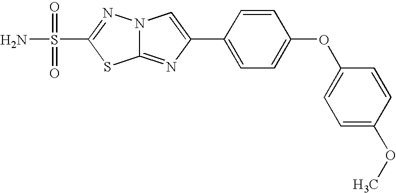 |
| 99 | 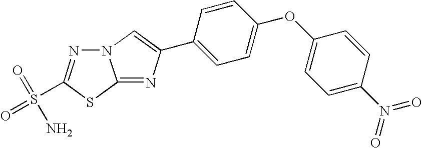 |
| 100 | 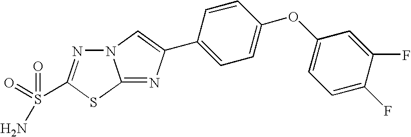 |
| 104 | 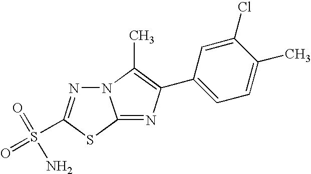 |
| 105 | 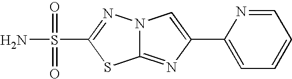 |
| 106 | 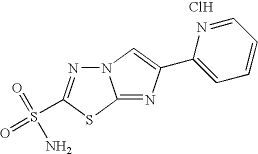 |
| 107 | 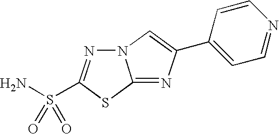 |
| 108 | 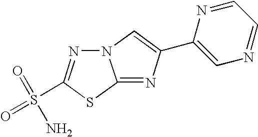 |
| 109 | 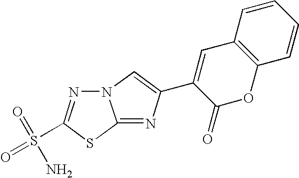 |
| 111 | 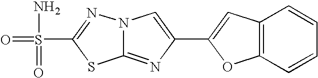 |
| 112 | 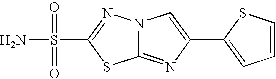 |
| 113 | 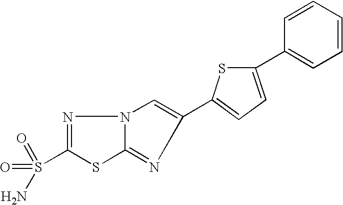 |
| 114 | 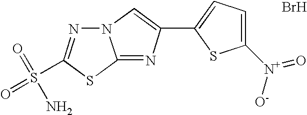 |
| 115 | 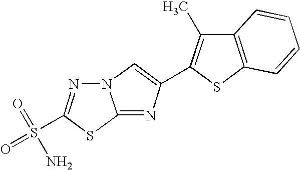 |
| 116 | 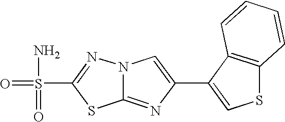 |
| 117 | 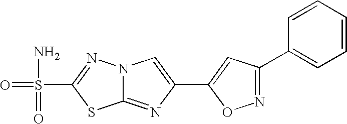 |
| 118 | 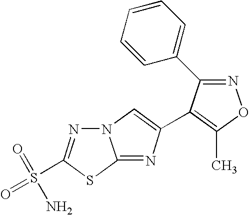 |
| 120 | 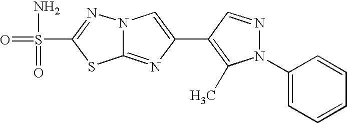 |
| 121 | 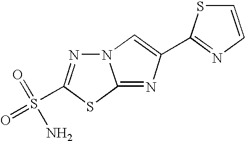 |
| 122 | 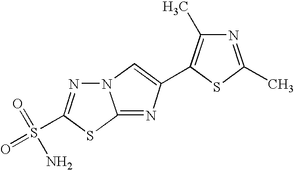 |
| 123 | 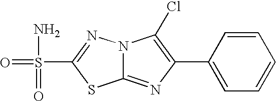 |
| 124 | 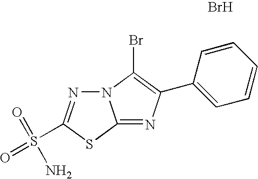 |
| 125 | 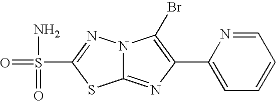 |
| 126 | 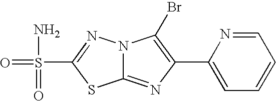 |
| 127 | 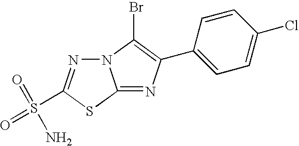 |
| 128 | 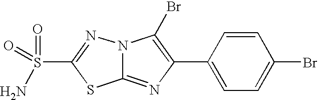 |
| 129 | 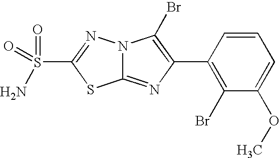 |
| 130 | 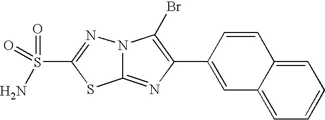 |
| 131 | 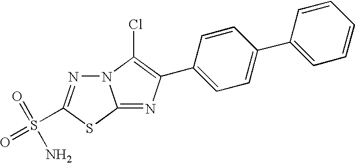 |
| 132 | 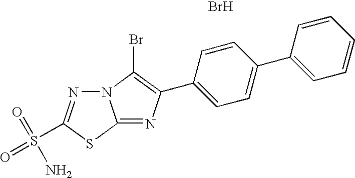 |
| 133 | 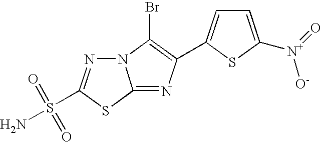 |
| 134 | 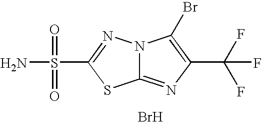 |
| 135 | 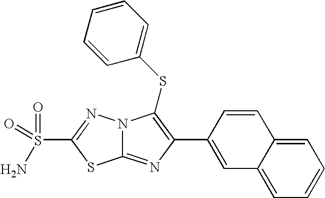 |
| 136 | 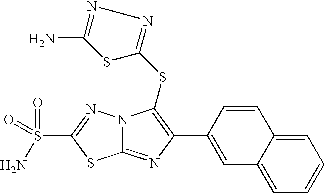 |
| 137 | 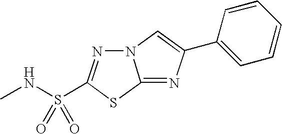 |
| 138 | 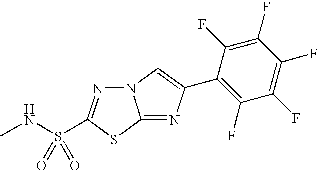 |
| 139 | 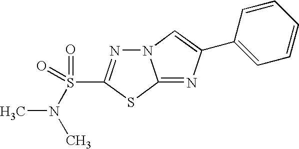 |
| 140 | 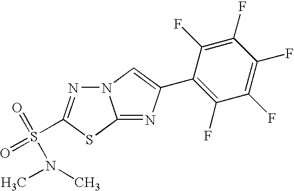 |
| 141 | 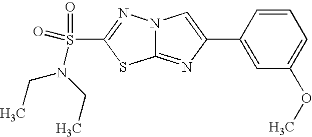 |
| 143 | 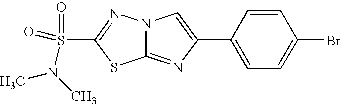 |
| 144 | 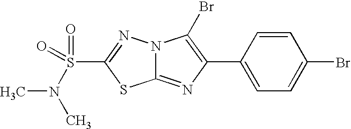 |
| 145 | 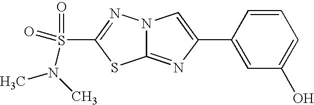 |
| 146 | 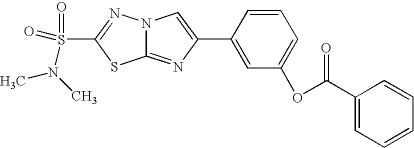 |
| 147 | 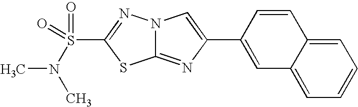 |
| 148 | 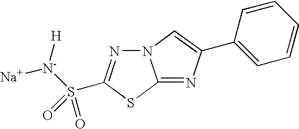 |
| 149 | 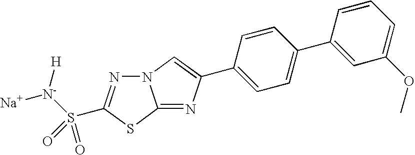 |
| 150 | 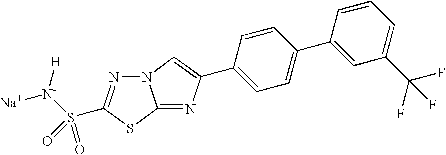 |
| 153 | 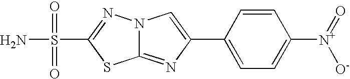 |
| 154 | 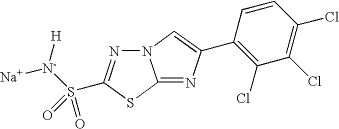 |
| 155 | 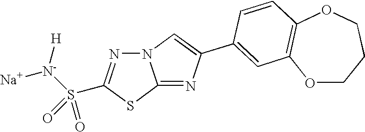 |
| 156 | 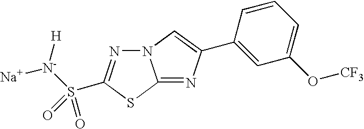 |
| 157 | 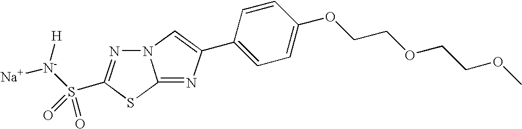 |
| 158 | 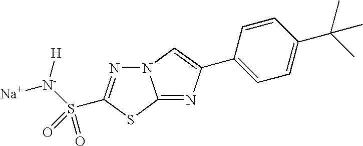 |
| 159 | 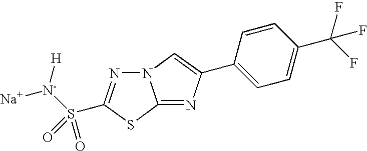 |
| 160 | 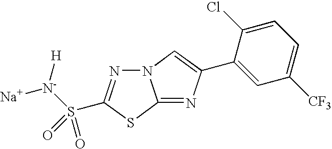 |

Other specific examples include compounds of Formula Ia:

| | |
|---|---|
| | Ia |
| 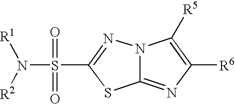 | |

| R¹ | R² | R⁵ | R⁶ |
|---|---|---|---|
| CH₃C(O)— | H | H | Ph |
| CH₃CH₂CH₂C(O)— | H | H | Ph |
| tert-BuOC(O)— | H | H | -Ph |
| Boc(H)NCH₂C(O)— | H | H | -Ph |
| TFA•H₂NCH₂C(O)— | H | H | -Ph |
| Ac(H)NCH₂C(O)— | H | H | -Ph |
| 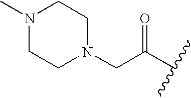 | H | H | -Ph |
| HO₂CCH₂CH₂C(O)— | H | H | -Ph |
| 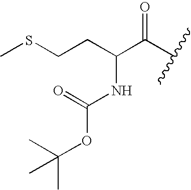 | H | H | -Ph |
| 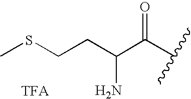 | H | H | -Ph |
| 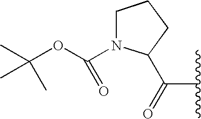 | H | H | -Ph |
| 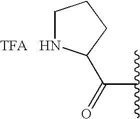 | H | H | -Ph |
| (CH₃)₂NCH₂C(O)— | H | H | 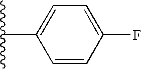 |
| CH₃C(O)— | H | H | 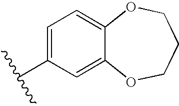 |
| CH₃OCH₂C(O)— | H | H | 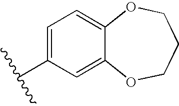 |
| CH₃CH₂CH₂C(O)— | H | H | 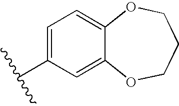 |
| CH₃C(O)— | H | H | 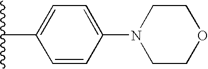 |
| CH₃OCH₂C(O)— | H | H | 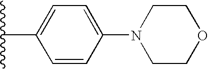 |
| CH₃CH₂CH₂C(O)— | H | H | 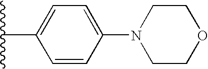 |
| CH₃C(O)— | H | H | 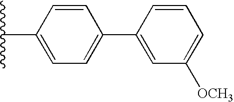 |
| CH₃OCH₂C(O)— | H | H | 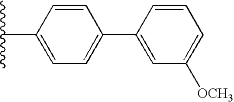 |
| CH₃CH₂CH₂C(O)— | H | H | 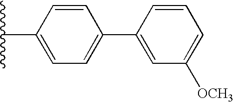 |
| CH₃O(O)— | H | H | 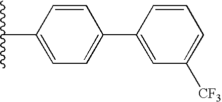 |
| CH₃CH₂CH₂C(O)— | H | H | 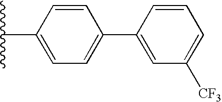 |
| CH₃OCH₂C(O)— | H | H | 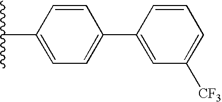 |
| CH₃CH₂CH₂C(O)— | H | H | 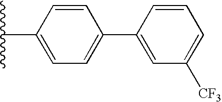 |
| 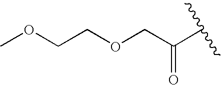 | H | H | 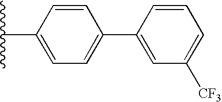 |
| 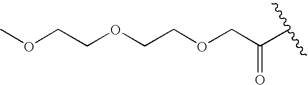 | H | H | 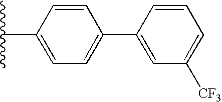 |
| tert-BuOC(O)— | H | H | 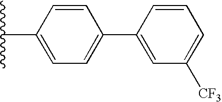 |
| CH₃C(O)— | H | H | 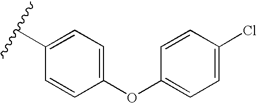 |
| CH₃OCH₂C(O)— | H | H | 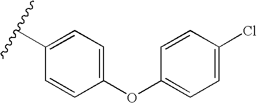 |
| CH₃CH₂CH₂C(O)— | H | H | 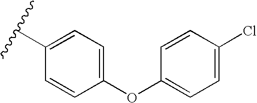 |
| 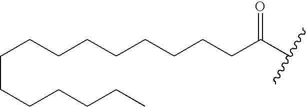 | H | H | 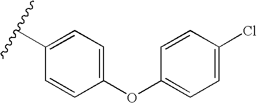 |
| PhCH₂OC(O)— | H | H | 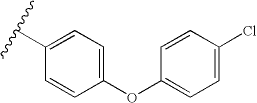 |
| 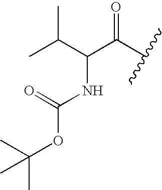 | H | H | 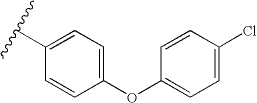 |
| 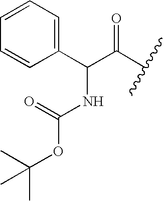 | H | H | 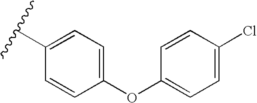 |
| 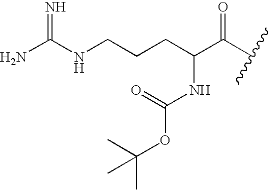 | H | H | 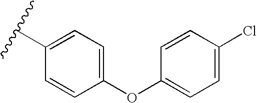 |

Other imidazo thiadiazole compounds which may be useful in practicing the methods of the present invention include:

| Compound Name | Structure |
|---|---|
| imidazo[2,1-b]-1,3,4-thiadiazole-2- sulfonimide | 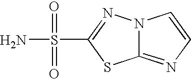 |
| 5-phenylimidazo[2,1-b]-1,3,4- thiadiazoie-2-sulfonamide | 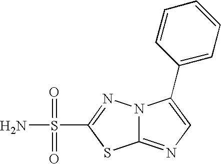 |
| 6-(1,1-dimethylethyl)imidazo[2,1-b]- 1,3,4-thiadiazole-2-sulfonamide | 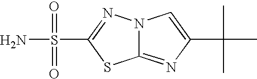 |
| 6-(2-furanyl)imidazo[2,1-b]-1,3,4- thiadiazole-2-sulfonamide | 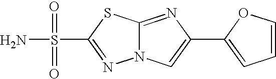 |
| 5-bromo-6-(2-furanyl)imidazo[2,1-b]- 1,3,4-thiadiazole-2-sulfonamide | 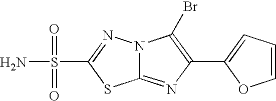 |
| 2-(aminosulfonyl)-6- phenylimidazo[2,1-b]-1,3,4- thiadiazole-5-carboxylic acid ethyl ester | 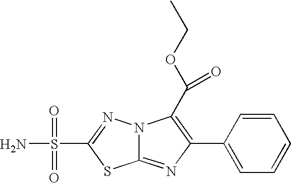 |
| 6-[(4-oxo-3(4H)- quinazolinyl)]methylimidazo[2,1-b]- 1,3,4-thiadiazole-2-sulfonamide | 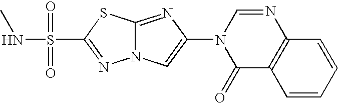 |
| 6-(5-(4-nitrophenyl)-2- furanyl)imidazo[2,1-b]-1,3,4- thiadiazole-2-sulfonamide | 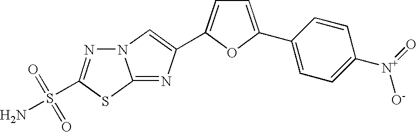 |
| 5-bromo-6-(5-(4-nitrophenyl)-2- furanyl)imidazo[2,1-b]-1,3,4- thiadiazole-2-sulfonamide | 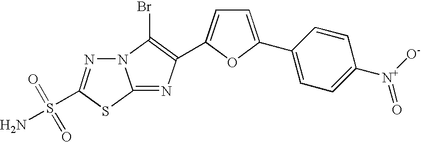 |
| 5-bromo-6-(2-oxo-2H-1-benzopyran- 3-yl)imidazo[2,1-b]-1,3,4-thiadiazole- 2-sulfonamide | 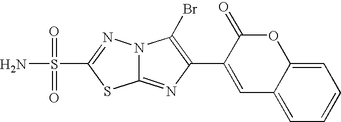 |

### 2. Compositions

The compounds of the present invention, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions of the invention with an appropriate pharmaceutically acceptable carrier, diluent or excipient, can be prepared by mixing a compound of the present invention, with the carrier, diluent or excipient and then may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of neuropathic pain as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is typically in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, a composition may contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, typically physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is typically sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains at least 0.01% by weight of the compound of the present invention.

The pharmaceutical composition of the present invention may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the present invention of at least 0.1% w/v (weight per unit volume).

The pharmaceutical composition of the present invention may be used for rectal administration in the form of for example, a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the present invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical composition of the present invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine specific aerosols.

The pharmaceutical compositions of the present invention may be prepared by methodology well known in the pharmaceutical art. For example, a pharmaceutical composition intended to be administered by injection can be prepared by mixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the neuropathic pain, and the subject undergoing therapy.

### 3. Utilities

The acylated and non-acylated imidazo[2,1-b]-1,3,4-thiadiazole-2-sulfonamide compounds have now been discovered to provide either treatment and/or prophylaxis of neuropathic pain. Thus, the compounds and pharmaceutical compositions described herein find use as therapeutics for treating and/or prophylaxis of neuropathic pain in mammals, particularly humans.

As discussed above, the compounds described herein are suitable for use in a variety of drug delivery systems. Injection dose levels for treating pain related conditions may range from about 0.1 mg/kg to about 10 mg/kg by an intravenous route. An intramuscular injection regimen may deliver the amount in one to three daily doses. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the treatment of long-term conditions, such as chronic neuropathic pain, the regimen for treatment may stretch over many months or years so oral dosing is typical for patient convenience and tolerance. With oral dosing, one to five and especially two to four and typically three oral doses per day may be representative regimens. Using these dosing regimens, each dose may provide from about 0.1 to about 100 mg/kg of the compound, with typical doses each providing from about 0.1 to about 50 mg/kg.

The compounds can be administered as the sole active agent or they can be administered in combination with active analgesic agents, such as opioid analgesic agents, including morphine, tramado, buprenorphine, pethidine, oxycodone, hydrocodone and diamorphine, paracetamol, gabapentin, aspirin and the NSAIDs.

Also useful in combination therapy with compounds of the present invention are agents from the antidepressant class such as, amitriptyline, desipramine, maprotiline, paroxetine, nortriptyline and venlafaxine; anti-convulsants such as carbamazepine, valproate, gabapentin and clonazepam; and local anesthetics such as mexiletine and lidocaine.

For the prophyaxis of neuropathic pain, the aforesaid compositions may also be administered to the subject.

### EXAMPLES

The following examples are for illustrative purposes only and are intended to be non-limiting.

### Synthesis of Compound 1:

### 6-Phenylimidazo[2,1-b]-1,3,4-thiadiazole-2-sulfonamide

2-Bromoacetophenone (4.00 g, 20.0 mmol) and 2-amino-1,3,4-thiadiazole-5-sulfonamide (3.60 g, 20.0 mmol) were refluxed in ethanol (150 mL) for 60 hrs. The resulting solution was cooled on ice and the resulting precipitate was collected by filtration and washed with ethanol to provide compound 1 as a white crystalline solid (2.50 g, 44%). ¹H NMR (200 MHz, DMSO-d⁶) δ 8.89 (s, 1H), 8.72 (br s, 2H), 7.90 (d, J=7.3 Hz, 2H), 7.43 (t, J=7.3 Hz, 2H), 7.32 (t, J=7.3 Hz, 1H).

### Synthesis of Compound 148:

### 6-Phenylimidazo[2,1-b]-1,3,4-thiadiazole-2-sulfonamide mono sodium salt

Compound 1 (200 mg, 0.71 mmol) was added to a solution of sodium hydroxide (28 mg, 0.71 mmol) in 4:1 MeOH/H₂O (5 mL). The solution was stirred overnight at room temperature. Volatiles were removed under reduced pressure to provide compound 148 as a white solid (235 mg, 99%). ¹H NMR (200 MHz, DMSO-d⁶) δ 8.59 (s, 1H), 7.85 (d, J=8.2 Hz, 2H), 7.32 (m, 3H).

### Pharmacokinetics

Compound may be delivered by various routes including, for example, IV, SC, intramuscular or oral. Various delivery routes and formulations are possible. For example, one soluble aqueous formulation involves the dissolution of the mono-sodium salt of a compound of in the instant invention in 20% HPCD, often buffered with sodium bicarbonate buffer. This soluble formulation is suitable for SC, IV, IM and oral administration of the drug, providing acceptable plasma concentration of drug.

Alternatively, compounds of the instant invention may be administered in their parent/non-ionized form either as a solid or dissolved in an appropriate solvent or excipient mixture.

In either case, it is the free base that is the active species and is quantified in vivo. For example, compound 1 represents the free base or parent form, while compound 148 is the mono sodium salt of compound 1. Compound 148 may be formulated in 20% HPCD and delivered SC to an animal, but once compound 148 dissociated from the 20% HPCD it is neutralized in the plasma and circulates in vivo as the free base, compound 1. Similarly, the deliver of compound 148 in 20% HPCD orally will result in the neutralization of compound 148 by stomach acids, and so compound 1 is absorbed by the subject.

By the methods similar to those described for compound 148, above, the following free bases may be converted to their corresponding mono sodium salts.

| Free Base (Cpd. #) | Na-Salt (Cpd. #) |
|---|---|
| 1 | 148 |
| 12 | 154 |
| 21 | 155 |
| 24 | 156 |
| 30 | 157 |
| 49 | 158 |
| 52 | 159 |
| 53 | 160 |
| 81 | 150 |

Compounds of the instant invention demonstrate acceptable pharmacokinetics when administered by various routes.

### Compounds of the Invention Reverse Conduction Velocity Deficits, Attenuate Axonal Atrophy, Ameliorate Neuropathic Pain in STZ Treated Diabetic Rats, and Prevent CFA-Mediated Hyperalgesia

We have previously shown that compounds of the present invention ameliorate neuronal cell death in vitro from NGF withdrawal or exposure to chemotherapy drugs. In vivo the compounds can attenuate chemotherapy-induced neuropathy induced by cisplatin, paclitaxel and oxaliplatin. The data presented here demonstrate that Compound 150 treatment to diabetic rats can ameliorate neuropathic changes in nerve conduction velocity (NCV) and axonal atrophy with chronic treatment (2 months). Furthermore, Compounds 155, 157, 154, 158 and 160 can reverse neuropathic pain in diabetic rats when given by subcutaneous and/or oral delivery routes. A unique feature of the analgesic effects is that the pharmacodynamic effect of the compounds takes approximately 3-6 hours to manifest and can last for up to 24 hours after a single administration (exemplified by Compounds 150 and 158), and with repeat administration, these effects can last for 24-48 hours. This is a very different profile from conventional therapies where the pharmacodynamic activity of the drug usually matches the plasma pharmacokinetics, resulting in efficacy of short duration and the necessity for frequent dosing.

In order to expand and verify the analgesic effects of this class of compounds, they were also tested in a Complete Freund's Adjuvant (CFA) model of hyperalgesia in rats. Compounds 150, 155, 157 and 158 were active after subcutaneous and/or oral delivery, effectively restoring pain sensitivity to normal in rats.

These results are summarized in the table below.

| Compound No. | DOSE | | ROUTE | REGIMEN | CFA | DN |
|---|---|---|---|---|---|---|
| 150 | 10 | mg/kg | sc | acute single | +ve (10 mg/kg) | +ve (10 mg/kg) |
| 155 | 1-10 | mg/kg | sc | acute single | +ve (3-10 mg/kg) | +ve (≦10 mg/kg) |
| 157 | 1-10 | mg/kg | sc | acute single | +ve (1-3 mg/kg) | <10 mg/kg |
| | 10-40 | mg/kg | po | acute single | +ve (20-40 mg/kg) | +ve (10-20 mg/kg) |
| | 5-20 | mg/kg | po | 5 d loading | +ve (5-10 mg) | +ve (5-10 mg) |
| 154 | 10 | mg/kg | sc | acute single | | +ve |
| 158 | 10 | mg/kg | sc | acute single | +ve (≦10 mg/kg) | +ve (≦10 mg/kg) |
| | 5-20 | mg/kg | po | 5 d loading | | +ve (5-10 mg/kg) |
| 160 | 10 | mg/kg | sc | acute single | | +ve 10 mg/kg |

The ability of these compounds to inhibit the JNK pathway and attenuate its activation represents a novel mechanism for addressing abnormal pain responsiveness in neuropathic conditions. Compound 150 represents a unique compound that impacts the underlying disease state of experimental diabetic neuropathy (conduction velocity deficits and axonal atrophy), and the class as a whole represents a novel approach to treating neuropathic or inflammatory pain states.

### The Effect of Compound 150 in Diabetic Neuropathy—Nerve Conduction Velocity & Degeneration

The effects of Compound 150 on nerve conduction (both motor and sensory) and axonal atrophy were examined in diabetic rats. A blinded reversal interventional paradigm was applied to evaluate two related small molecules on established experimental rat diabetic peripheral neuropathy of 2 months duration given over a subsequent 2 months, specifically evaluating motor and sensory conduction and sural axon caliber.

### Methods:

Male Sprague-Dawley rats (200-300 g) raised on sawdust covered plastic cages in a room with normal light dark timing and fed with standard rat chow were used for this experiment. The protocol was reviewed and approved by the University of Calgary Animal Care Committee adhering to the guidelines of the Canadian Council on Animal Care (CCAC). Diabetes was induced by a single intraperitoneal injection of streptozotocin (STZ) in citrate buffer (65 mg/kg) with age-matched controls given the buffer without STZ. Animals were used for the study if fasting glucose levels 5-7 days later were ≧16.0 mmol/L (One Touch FasTake strips, Johnson and Johnson).

Treatments were applied after 2 months of hyperglycemia for a duration of 2 months. Motor conduction recordings (1-3) were made prior to intervention then after one and two months of diabetes. Sensory conduction utilized the approach of Parry and Kozu involving stimulation of the digital branches of the sciatic nerve and recording from the sciatic nerve at the level of the popliteal fossa with near nerve temperature maintained at 37° C. (4).

At endpoint (4 months of diabetes, 2 months of treatment) the rats were euthanized and sural nerves harvested for morphometric studies. Sural nerves were fixed in cacodylate buffered glutaraldehyde, dehydrated with alcohols, fixed in osmium tetroxide then embedded in epon to generate one micron sections, as in previous work (5,6). Sections were photographed under oil immersion (1000×) to sample the entire sural nerve. Images were analyzed using Scion image offline to measure axon area for 100 myelinated axons for each sural nerve fascicle. Data consisted of arbitrarily and randomly selected 80 axons over 9 square microns in area (“large axons”) and 20 axons smaller than 9 square microns (“small axons”) in area. Surface areas generated by the calibrated Scion image analysis technique represent actual axon areas and are not corrected to a postulated circular shape, as occurs in some programs. Mean sural axonal areas were converted by a program generating estimates of circular axonal area from the axon circumference, an approach that generates larger mean sural areas (1,7,8). For sural nerves with more than one fascicle, each fascicle underwent separate analysis and a mean axon area was calculated for the rat from the fascicles. All measures were carried out with the experimentalist blinded to the treatment group.

For statistical analysis, we studied mean values with standard errors of the means and compared values in the interventional groups with one way ANOVA or repeated measures ANOVA and post hoc Student's t-tests.

### Results

(i) SNCV: Within Comparisons (Diabetic Groups Only): The Vehicle treated diabetic group had a significant reduction in SNCV from Baseline to 2 months post (p=0.005). Compound 150 treated groups did not significantly change from baseline. Thus, while diabetic animals worsened, the drug treated animals had stable SNCV over the same time period.

Between Group Comparisons (Diabetics v Normals) Compound 150 (5 days per week) treated diabetic animals were not significantly different from normals treated with Compound 150 after 2 months of treatment (p>0.05), demonstrating that Compound 150, dosed 5 days per week, reversed the effects of diabetes on SNCV in diabetic rats. Compound 150, dosed 2 days per week, did not confer similar protection as diabetic rats were significantly different from both normal animals treated with vehicle or compound 150.

Between Group Comparisons (Diabetics ONLY): At baseline: All diabetic groups were equivalent. At 2 months: Animals receiving compound 150 (5 days/week) were significantly better than vehicle treated diabetics (p=0.04). Compound 150 given twice per week did not afford similar protection.

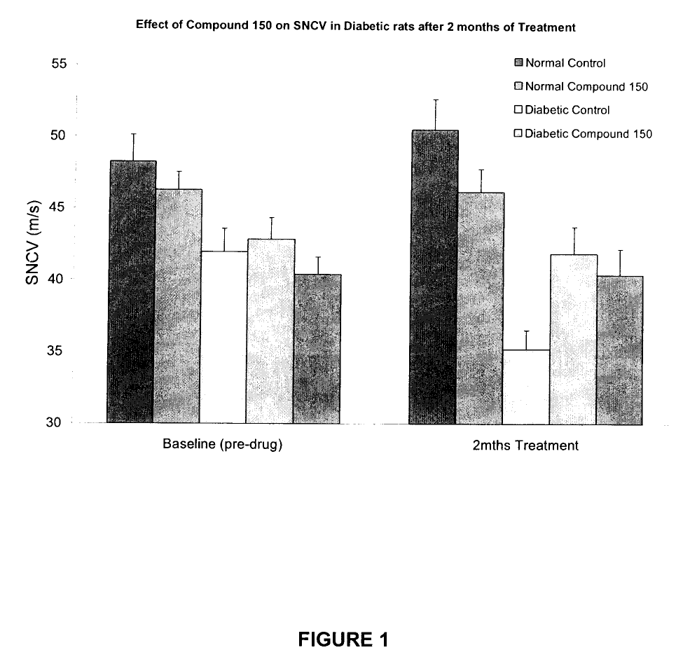

Results are illustrated in FIG. 1.

(ii) MNCV: Within Comparisons (Diabetic groups only): There was no change in the diabetic control group over time. Compound 150 (5 days per week) caused a significant improvement in MNCV from baseline to 2 months in diabetic animals (p=0.007).

Compound 150 (2 days per week) had identical effects as the drug given 5 days per week in diabetic rats (p=0.005 & 0.001 respectively).

Between Group Comparisons (Diabetic v Normals): Compound 150 (5 days per week) did not restore MNCV to normal in diabetic rats (compared to normals similarly treated with Compound 150). Compound 150 (2 days per week) did not restore MNCV to normal (compared with Vehicle treated normals and normals treated with Compound 150 of 5 days per week.

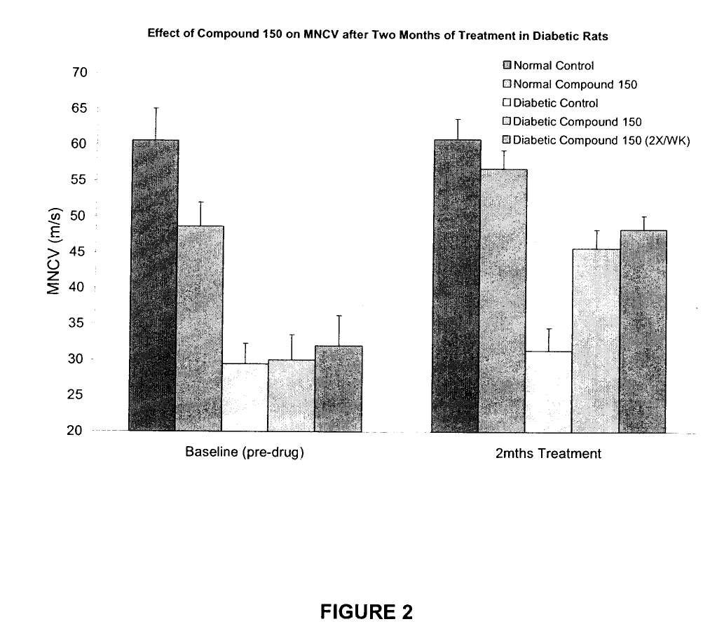

Between Group Comparisons (Diabetics ONLY): Baseline: All diabetic groups were equivalent. 2 months: Animals receiving Compound 150 (5 days per week and 2×/week) were significantly better than vehicle treated diabetics respectively; p=0.007 & 0.002). Results are illustrated in FIG. 2.

(iii) Sural nerve myelinated axon morphometry: Morphometric studies were confined to nondiabetics and diabetics given Compound 150 (5 of 7 days) or vehicle so as to analyze changes in those with more robust electrophysiological changes. For mean area of all measured axons in all 4 groups, ANOVA was not significant but separate analysis (two tailed Student's t-test) comparing only diabetics given vehicle vs. those given Compound 150 identified a rise in mean axonal area with the active agent (p=0.016). Only a nonsignificant trend toward smaller mean area was observed when comparing nondiabetics and diabetics given vehicle. Comparison of mean axonal area in only “large” (greater than 9 microns squared area) myelinated axons was next carried out. ANOVA among the four groups was not significant. As in the above analysis, however, separate comparison (two tailed Student's t-test) between diabetics receiving vehicle vs. Compound 150 noted a significant increase in mean axonal area with the active agent (p=0.012), As above, there was only a nonsignificant trend toward smaller mean area when comparing nondiabetics and diabetics given vehicle.

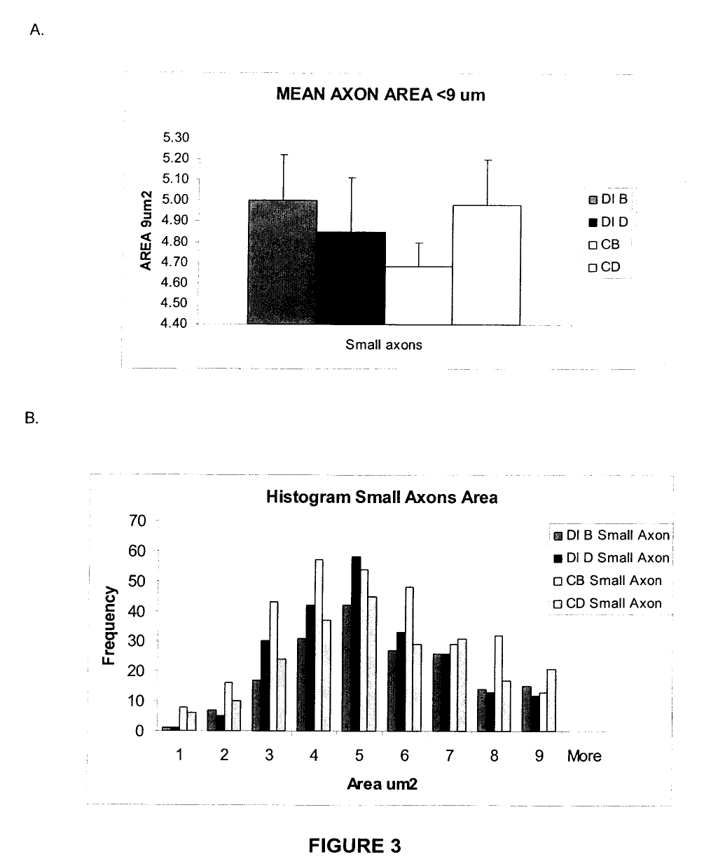

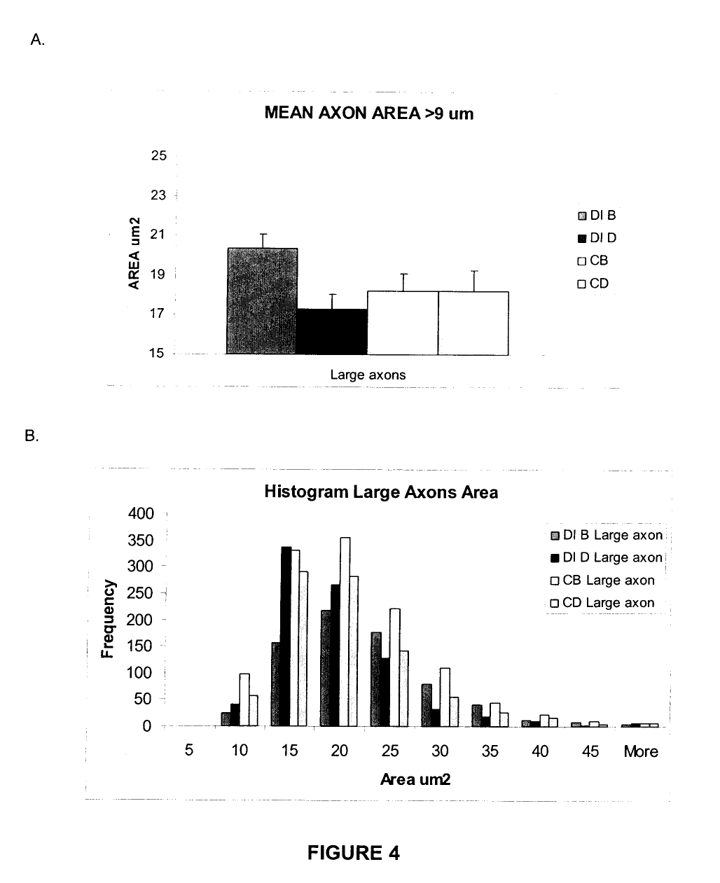

Results are given in FIGS. 3 and 4.

### Discussion

An experimental model of Type I diabetic neuropathy in rats was used. Rats exposed to 2 months of experimental diabetes subsequently treated for 2 months with Compound 150 5/7 days weekly exhibited benefits in motor and sensory nerve conduction velocity compared to those treated with vehicle alone. Sural myelinated axons in rats treated with Compound 150 5/7 days had larger areas than those given vehicle alone. The findings identify impact of Compound 150 on three indices of experimental diabetes.

Human diabetic polyneuropathy (DPN), associated with sensory loss, pain and heightened risk of foot amputation, is common (50% of diabetic subjects) and disabling. No treatment is available to arrest or reverse the disease. Sensory involvement is the earliest and most prominent form of the disease in humans, but later motor weakness may also develop. Several experimental models exist to test novel forms of therapy but the most common studied and reported is that associated with streptozotocin (STZ) in rats. STZ is a beta cell toxin that is associated with the abrupt onset of hyperglycemia in 3-5 days and is used as a model of Type I human disease. Rats given STZ survive out through 12 months and beyond without the requirement for insulin. Without insulin, the model allows more rapid analysis of the development of DPN without the problem of potential confounding neurotrophic properties of insulin. There is a large literature on interventional approaches to using this model in developing human therapeutics. Several caveats have emerged in using the model that can improve its value in predicting future human therapy. While many studies show motor conduction slowing, a hallmark electrophysiological feature of the disease, such slowing occurs very early in the model and is malleable to a large number of approaches reported. It also may not reflect direct sensory involvement in diabetes. More rigorous interventional approaches emphasize: (i) recordings of motor and sensory (caudal nerve, or more recently sciatic digital nerves) conduction under strict near nerve temperature control; (ii) a “reversal” paradigm such that intervention is applied after there is already established diabetes and features of DPN; (iii) a model of sufficient duration (of final duration greater than 8 weeks) to better reflect translation of model information to human disease where DPN develops over decades; (iv) adding additional indices of DPN as endpoints in the study (e.g. sural nerve morphometry, epidermal fiber innervation, tactile allodynia). While the STZ rat model of diabetes does not demonstrate overt dropout of axons in the sciatic or sural nerves or loss of sensory neurons in ganglia, there is atrophy of sural nerve axons (if the duration of diabetes is at least 2-3 months), and loss of skin epidermal axons. We have suggested that overall the rat STZ model is valuable in modeling early features of human DPN that do not include catastrophic neuron loss. As such the model illustrates a unique pathophysiological process: retraction of the terminal fibers first in target organs (e.g. skin) with retrograde atrophy of axons, concurrent changes in excitability (conduction velocity), downregulation of gene expression in sensory neurons of structural and other proteins destined for axons (with upregulation of some survival and injury molecules) and only much later eventual dropout of neurons or axons. In STZ rats dropout does not occur out to 12 months of diabetes.

Hyperglycemia was associated with robust electrophysiological features of DPN by 2 months slowing of motor and of sensory conduction velocity. As discussed above, sural nerve myelin thinning and frank axon dropout are not features of this model. Axon atrophy, however, may be observed in some studies of this duration using this model but is generally mild. Atrophy represents a decrease in mean axonal area or diameter. In this study sural axon areas trended toward lower values in diabetics treated with vehicle compared to nondiabetics but the difference did not achieve statistical significance.

Compound 150 initiated at 2 months of established DPN reversed slowing of both motor and sensory conduction velocity. None of the interventions normalized slowing and no trend toward improvement was observed after only one month of treatment. None of the agents exhibited evidence of neurotoxicity. Compound 150 showed the most robust improvements and was chosen for morphometric work. A direct comparison of diabetics treated with vehicle vs. agent indicated increased axonal area in the diabetics receiving Compound 150.

In evaluating potential new compounds destined for possible translation into human DPN studies, most recent clinical trials have relied on preclinical nerve conduction data. There have been a large number of interventions in the STZ rat model identifying a rise in motor conduction velocity. A number, however, can be criticized as evaluating very short term experimental diabetes, as applying intervention from the outset of hyperglycemia (prevention paradigm) or of relying only on motor conduction results. In the current work, the approach reversed established electrophysiological abnormalities and there were concurrent changes in motor and sensory axons. The identification of a rise in axonal caliber in the cohort treated Compound 150, albeit mild (and with only a trend toward atrophy in the diabetic group) is important because mild atrophy can be demonstrated in this model of similar duration and its reversal with other approaches (e.g. intrathecal insulin) paralleled electrophysiological improvement as well. Atrophy most likely reflects an impairment of neuronal synthesis, export and insertion of neurofilaments into axonal segments (5). While axonal atrophy can generate slowing of conduction in axons, its development in diabetes likely represents a different, structural facet of the disease. Conduction slowing develops rapidly in STZ diabetes before atrophy or declines in neurofilament export can be identified. More likely it reflects a metabolic induced change in axon excitability as described by Sima and colleagues (12). Thus, the aforesaid results identify three separate impacts of the compounds on experimental DPN: motor conduction, sensory conduction and axon caliber.

Treating Neuropathic Pain Associated with Diabetic Neuropathy

The effects of compounds 150, 155, 157 and 158 on neuropathic pain responses characterized by tactile allodynia in diabetic rats were examined. A blinded reversal interventional paradigm was applied to evaluate the compounds, with therapy initiated when an aberrant pain state was clearly established. The effects of single or repeat (5 or two days per week) dosing regimen were assessed as described.

### Methods:

Rats (female Sprague Dawley; 250-270 g) were rendered diabetic with the commercially available agent streptozotocin and, were compared to vehicle-treated age matched controls, maintained for up to 6 weeks or more. Standard physiologic parameters (body weight and blood glucose) were recorded before, during and after the study to assess the metabolic status of animals.

Study 1: Both normal and diabetic groups were divided into two groups of 12 and received either vehicle or Compound 150 in 20% HPCD (10 mg/kg, sc) 5 days per week, for two weeks. Standard indices of sensory nerve function (tactile response threshold) were measured at baseline, prior to drug treatments, 48 hours after the 5^{th} dose, and again prior to sacrifice (after the 10^{th} dose) along with the standard physiologic parameters of body weight and plasma glucose.

Study 2: As per Study 1, except animals were treated with either compound 157 or 158 in 20% HPCD (10 mg/kg, sc) for 14 consecutive days.

Study 3: After 1 month of diabetes rats were treated subcutaneously with a single administration of 150, 155, 157, 154, 158 or 160 in 20% HPCD, as indicated; orally by gavage with a single administration of 157, or for 5 consecutive days by oral gavage with 157 and 158 to assess cumulative effects. The effect of the compounds was assessed 6 hours after the single or final administration.

Detailed methods for performing the behavioral tasks can be found in Journal of Neuroscience Methods (1994), 53: 55-63 and Methods in Molecular Medicine, Volume 99: Pain Research: methods and protocols, edited by Z. D. Luo, Humana Press Inc., Totowa, N.J.

### Results:

### Study 1:

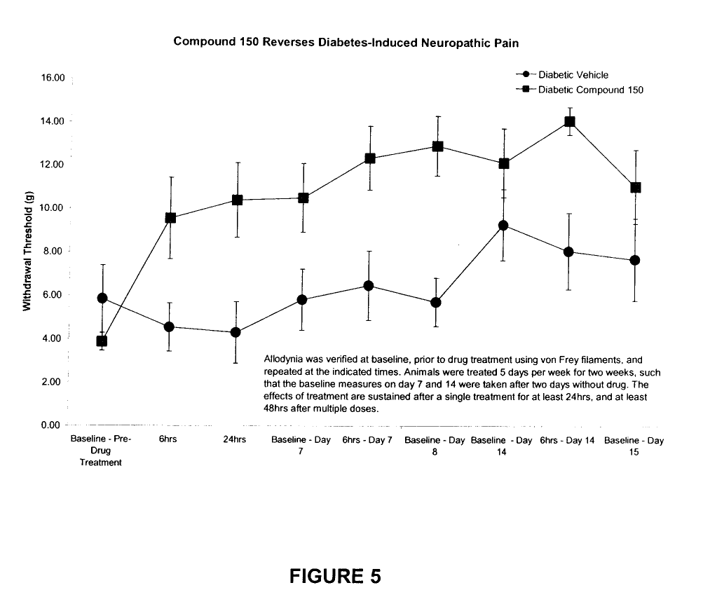

Animals were tested for tactile allodynia prior to and after 1, 5 and 10 injections with vehicle or Compound 150. The results are shown in FIG. 5. Diabetic animals demonstrated marked allodynia at baseline (FIG. 5), with lower response thresholds to von Frey filaments applied to the plantar surface of the hind paws. Six hours after the initial treatment with Compound 150, tactile allodynia was reversed in diabetic animals. This effect persisted throughout the remainder of the experiment, (FIG. 5)

Conclusions: Compound 150 had a marked effect on diabetes-induced neuropathic pain, indicated by the reversal in allodynia. The drug had a very different profile than a typical analgesic and likely has a very unique mechanism for affecting pain. Most straightforward analgesics have a rapid onset, and short period of action. After an initial injection to diabetic rats, Compound 150 took four to six hours to have an impact on pain, and this persisted for at least 24 hours. Multiple dosing had diabetic animals consistently responding within the normal range to tactile stimulation.

### Study 2:

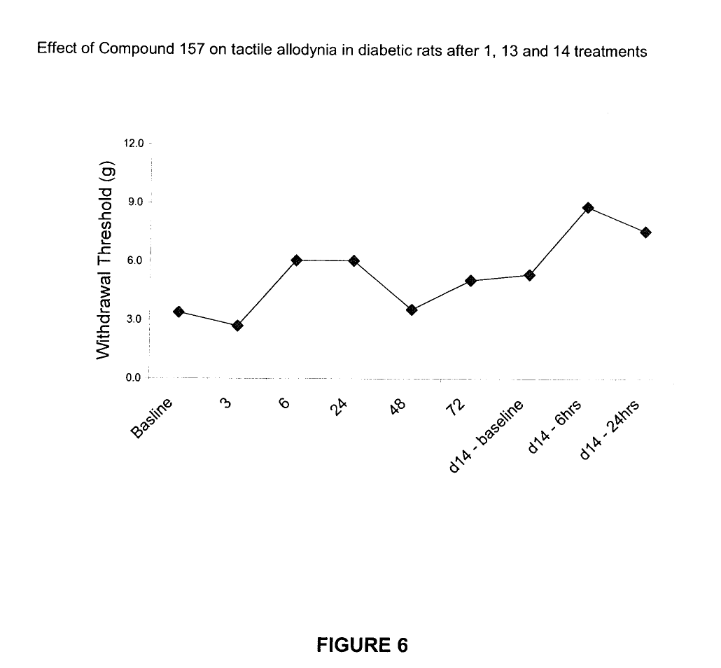

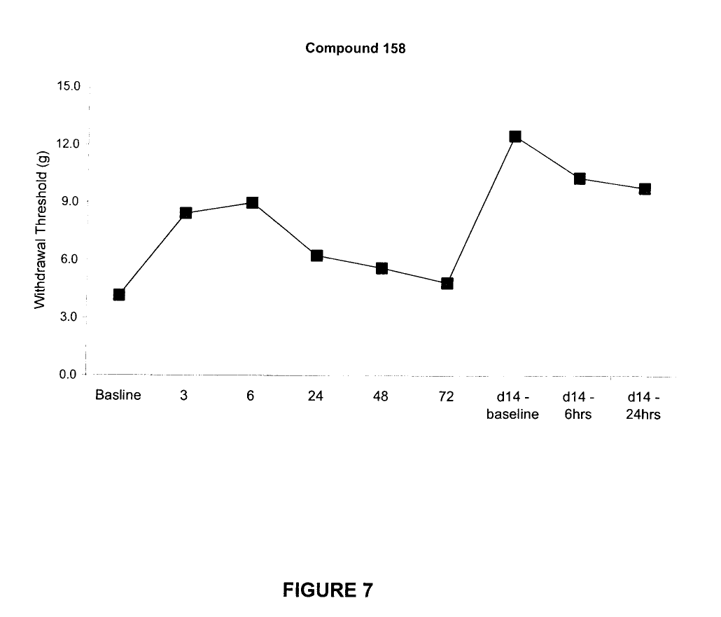

Compounds 157 (FIG. 6) and 158 (FIG. 7) demonstrated a rapid effect on tactile allodynia in diabetic rats starting from 3-6 hours after the initial treatment, with the effect of 157 persisting for 24 hours after a single administration. Like compound 150 in Study 1, with repeated dosing this effect was apparent for at least 24 hours after dosing for both 157 and 158 (the last time point assessed in the study) (FIGS. 6-7).

Conclusions: Both Compounds 157 and 158 reversed an established neuropathic pain state in diabetic rats. These compounds appear to offer an advantage over that reported for Gabapentin, since with repeat dosing there is a long lasting effect on neuropathic pain, which suggests better efficacy with a less frequent dosing requirement.

### Study 3:

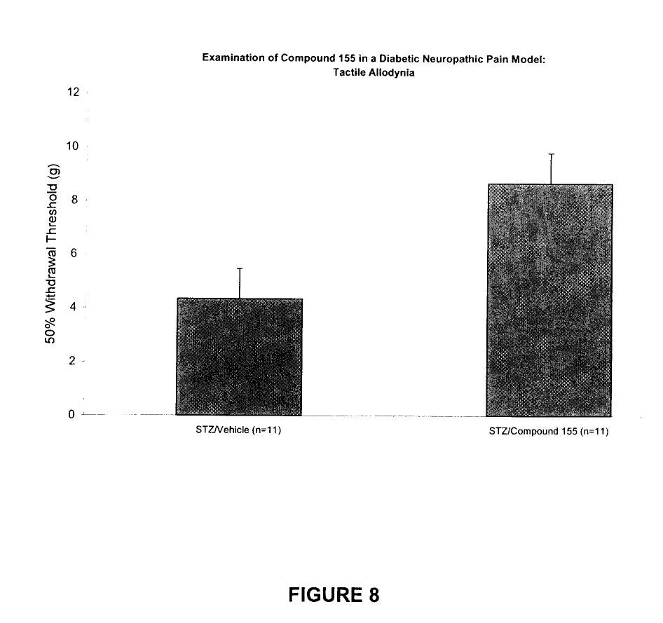

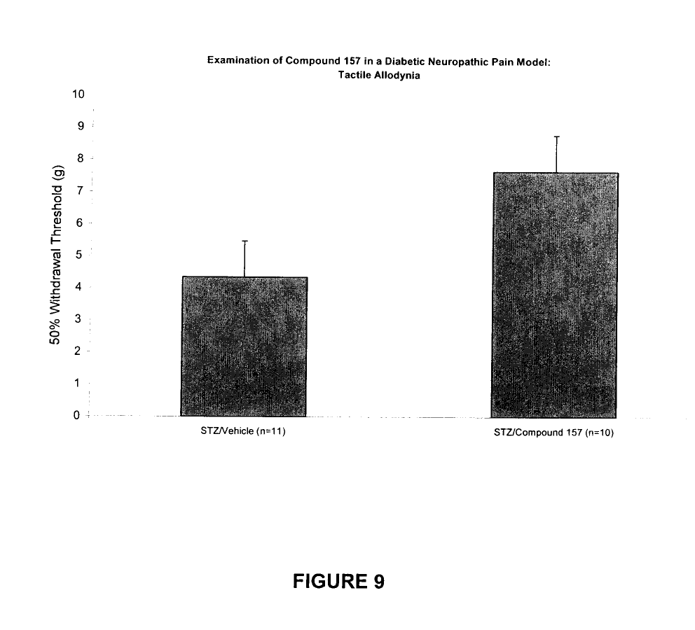

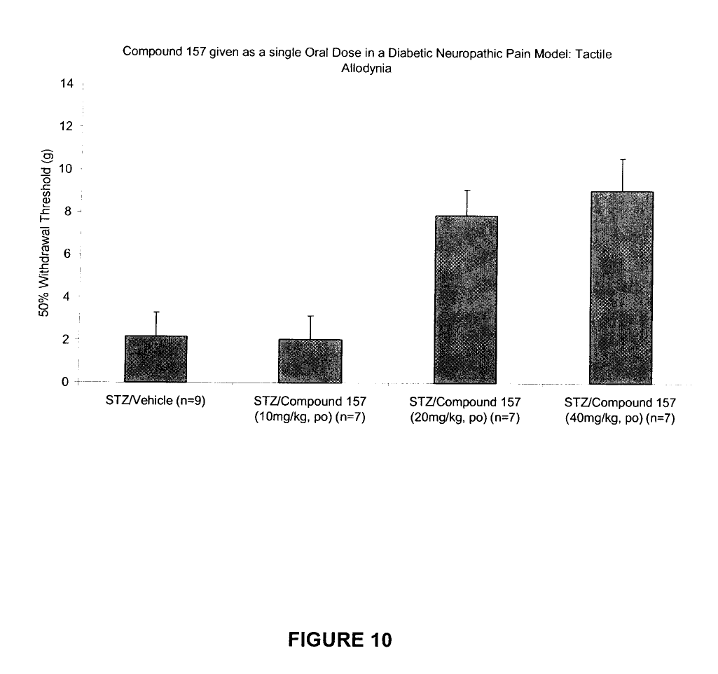

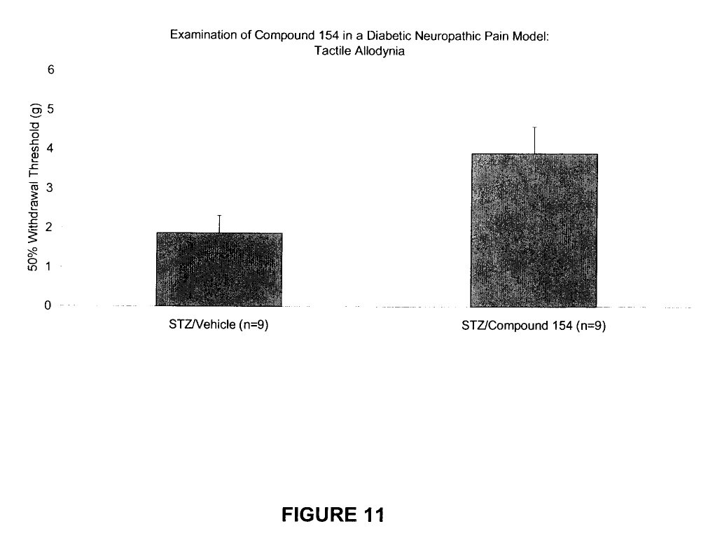

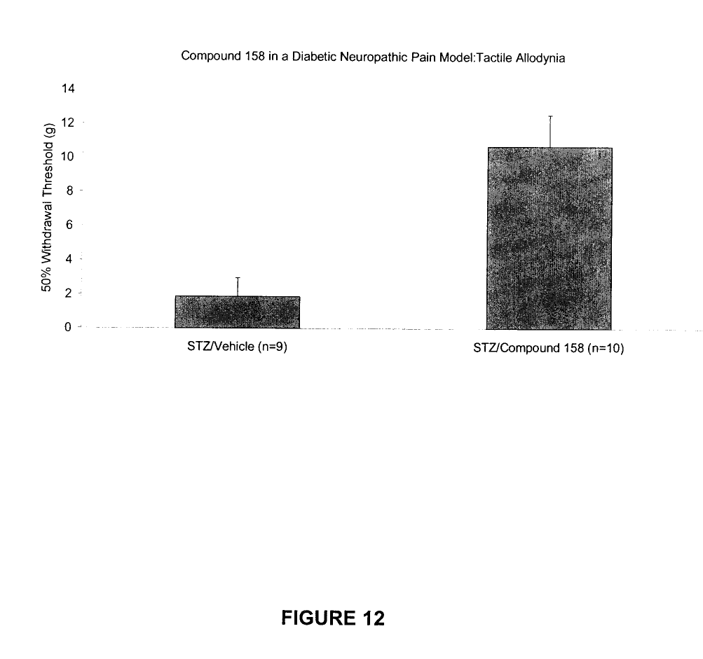

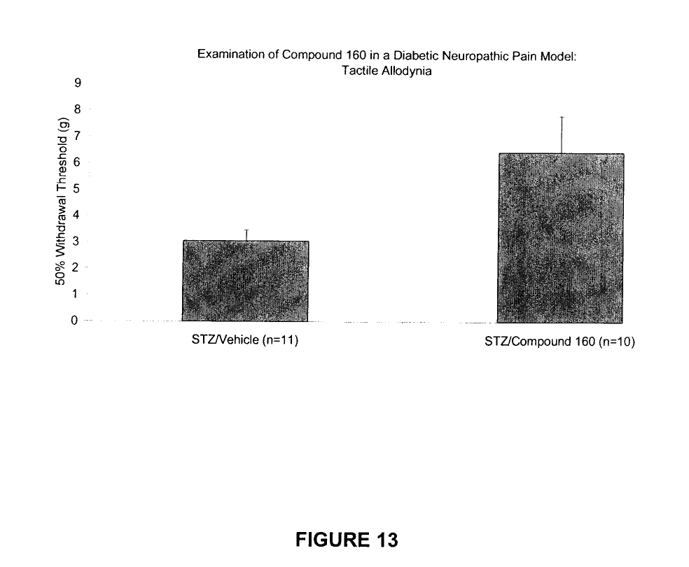

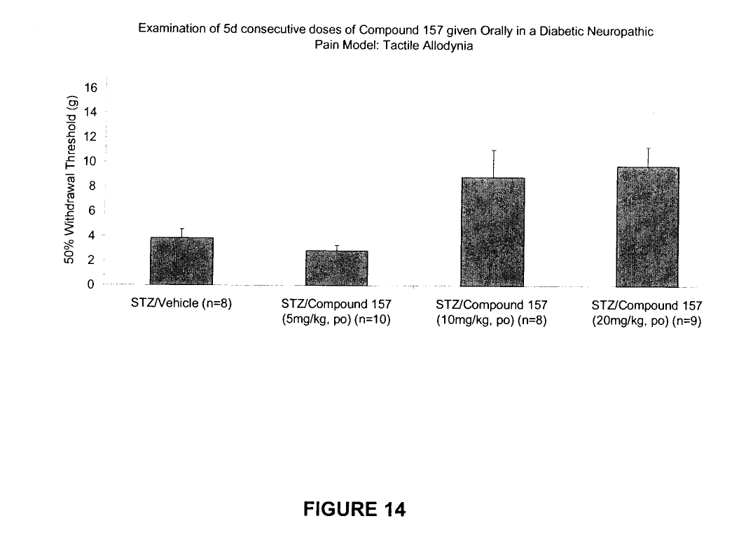

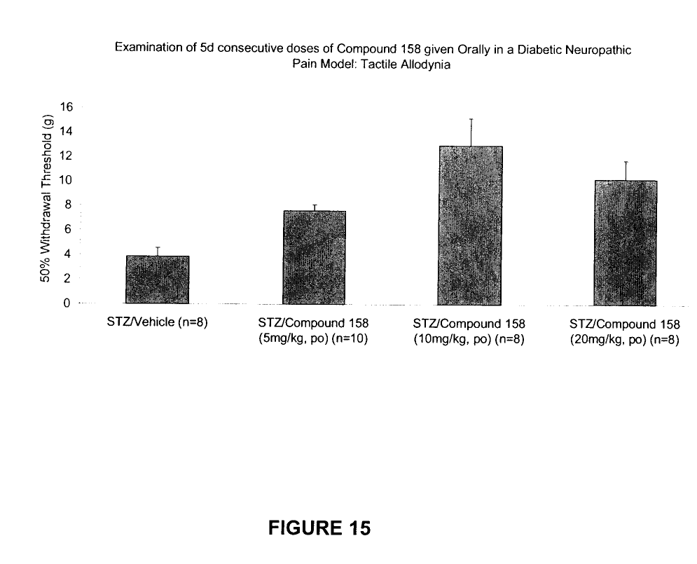

Effects of single administrations were observed for Compounds 150, 155, 157 (given both sc and po), and 154, 158, and 160 when examined 6 hours after a single administration to diabetic rats (FIG. 8-13, respectively). When 157 and 158 were dosed for 5 consecutive days by an oral route, equivalent efficacy was observed (FIGS. 14-15), confirming oral activity for the compounds. Of note, while 157 was efficacious as a single dose at 10-20 mg/kg, po, with repeated dosing the required dose range for efficacy was reduced to 5-10 mg/kg, po.

Conclusions: A common feature of this class of compounds is their ability to reverse neuropathic pain as measured by tactile allodynia in diabetic rats. They are orally active, and have a prolonged anti-allodynic effect after cumulative dosing.

### Effect of Compounds on CFA-Mediated Pain

Complete Freund's Adjuvant (CFA) was used to induce an inflammatory response, resulting in hyperalgesia. This model was chosen as a second experimental paradigm to obtain direct evidence for activity of the compounds against pain states because of its link with the induction of aberrant JNK phosphorylation, and evidence that this signaling cascade appears to, at least in part, mediate the pain response in this model (Doya et al., 2005).

### Methods:

Female Sprague Dawley rats were given either vehicle or Compound 150 (10 mg/kg, sc), 155 (1-10 mg/kg, sc), 157 (1-10 mg/kg, sc; 10-40 mg/kg, po) or 158 (10 mg/kg, sc) 6 hours prior to pain testing (compounds 150, 155, 157, and 158 were dissolved in 20% HPCD at 1-10 mg/mL). Compound 157 was also tested under conditions of repeat dosing where it was given at 5-20 mg/kg, po for five consecutive days. Under all treatment conditions, a single injection of CFA (50 uL) was given into the plantar surface of the right hind paw 1 hour prior to pain testing (i.e., 5 hours after the final administration of compound). Immediately after the CFA injection, animals were placed in testing chambers with a wire mesh bottom to habituate. Standard von Frey filaments were used to assess tactile response thresholds. The left, un-injected paw served as a control. Fibers were applied in the manner described by Dixon (1980) using the up-down method. The 50% withdrawal threshold (in grams) was determined for each paw.

### Results:

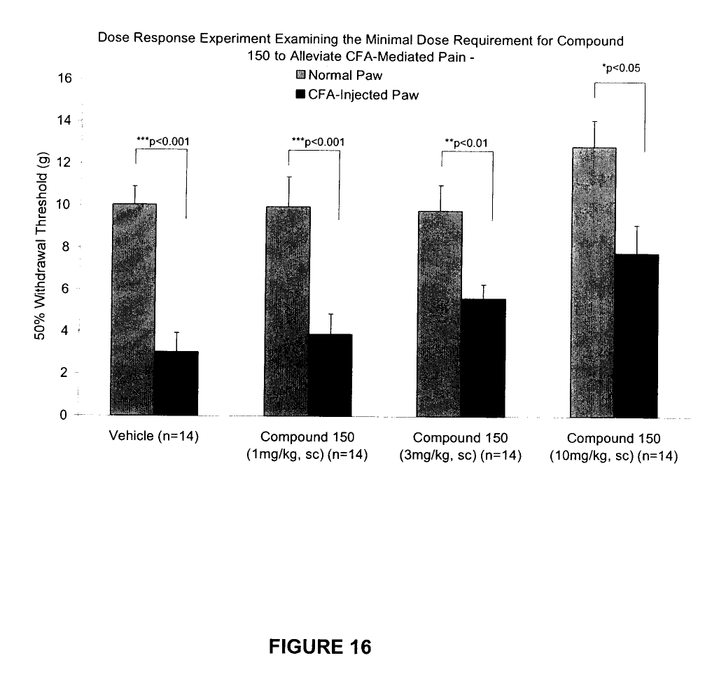

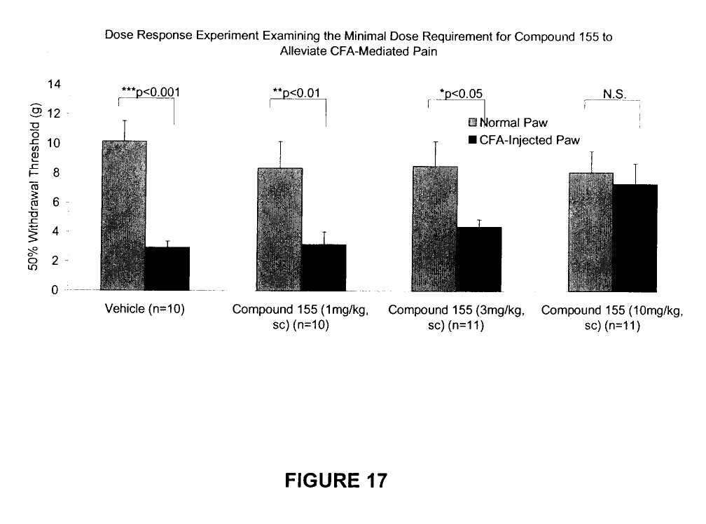

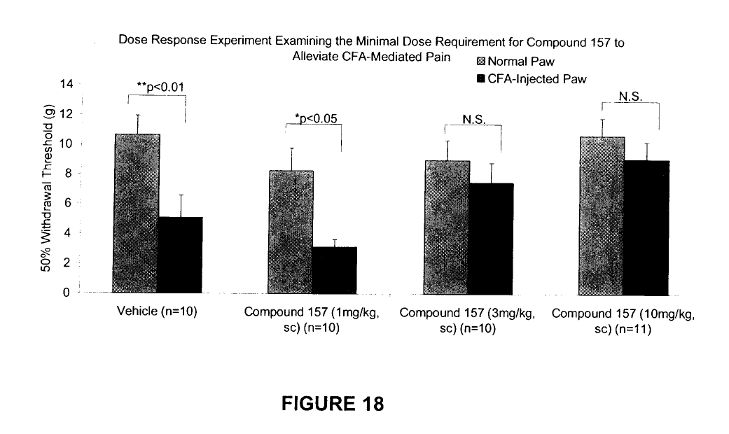

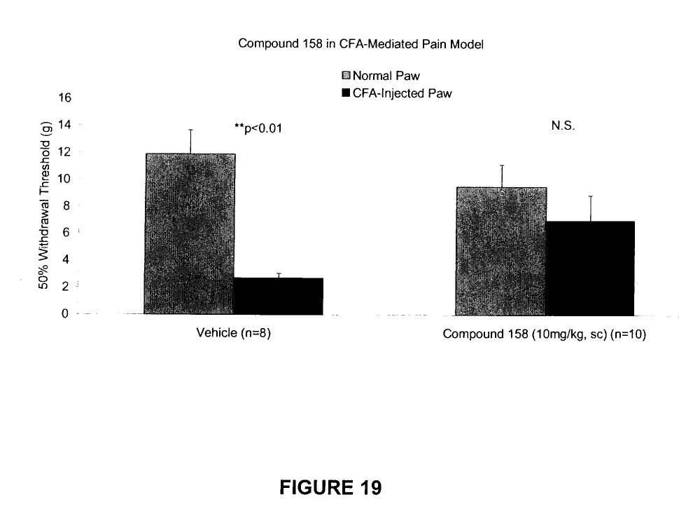

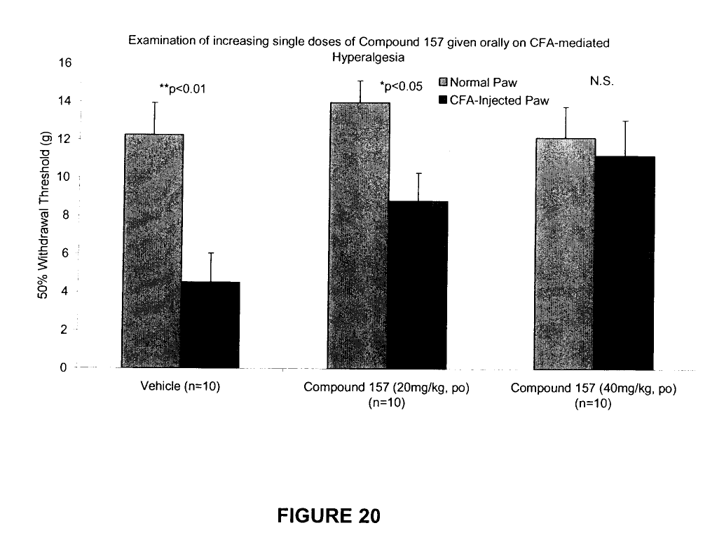

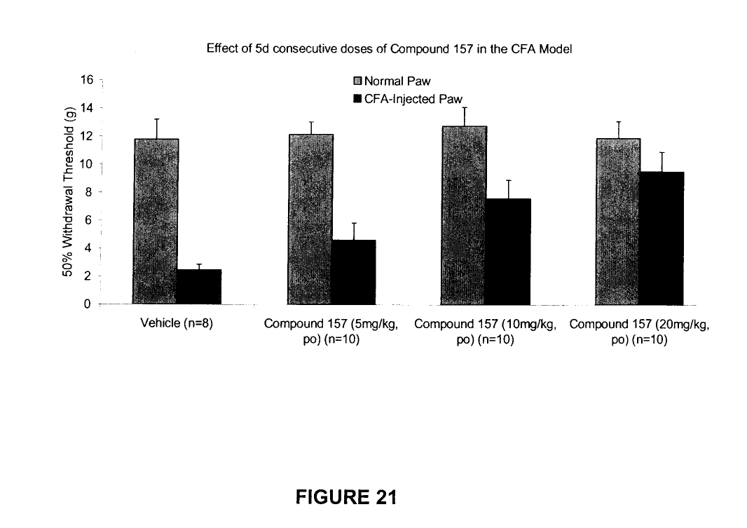

Compounds 150, 155, 157, and 158 all attenuated CFA-induced tactile hyperalgesia when given subcutaneously at doses≦10 mg/kg (FIGS. 16-19). Compound 157 was also tested orally in this model, and was efficacious in a dose range of 20-40 mg/kg, once again demonstrating oral activity (FIG. 20). However, if a repeat dose paradigm was applied with animals receiving daily dosing for 5 consecutive days, the required dose range was reduced to 5-10 mg/kg, po (FIG. 21).

Conclusions: This class of compounds shows robust efficacy in a second pain model, utilizing CFA to induce tactile hyperalgesia. Like in the STZ model, repeated drug delivery resulted in a lower dosing requirement.

### Overall Summary:

The compounds exemplified here are capable of impacting multiple facets of diabetes-induced neuropathy. In animals that have established conduction velocity deficits and neuropathic pain, these compounds were able to prevent the further decline (SNCV), or actually reversed (MNCV) conduction deficits, while attenuating tactile allodynia. Furthermore, neuronal atrophy was also favorably impacted by treatment, suggesting that these compounds are not just masking the symptomology of the neuropathy, but can favorably promote nerve health and function. The analgesic effects of the compounds translated to a second, inflammatory pain model, demonstrating that they likely have an impact on a common mechanism driving the different pain states. We believe this to be a novel mechanism which results from a drug-induced reduction in aberrant levels of phosphorylated JNK. Finally, another advantage of these compounds in the longevity of action, with effects seen for up to 24 hours, and in some cases 48 hours after repeated dosing. This might suggest that dosing frequency could be as little as once per day, or even once every other day. This offers clear advantage over current pharmaceuticals such as the opioids, and channel modulators, which require dosing multiple times per day, and not without significant side effects for many patients.

### REFERENCES

(1) Zochodne D W, Verge V M K, Cheng C, Sun H, Johnston J. Does diabetes target ganglion neurons? Progressive sensory neuron involvement in long term experimental diabetes. Brain 2001; 124:2319-2334.
(2) Brussee V, Cunningham A, Zochodne D W. Duplicate Use 15203 Direct insulin signalling of neurons revereses diabetic neuropathy. Diabetes 2004; 53(7):1824-1830.
(3) Zochodne D W, Ho L T. The influence of indomethacin and guanethidine on experimental streptozotocin diabetic neuropathy. Can J Neurol Sci 1992; 19(4):433-441.
(4) Parry G J, Kozu H. Piroxicam may reduce the rate of progression of experimental diabetic neuropathy. Neurology 1990; 40: 1446-1449.
(5) Scott J N, Clark A W, Zochodne D W. Neurofilament and tubulin gene expression in progressive experimental diabetes: failure of synthesis and export by sensory neurons. Brain 1999; 122:2109-2118.
(6) Brussee V, Cunningham F A, Zochodne D W. Direct insulin signaling of neurons reverses diabetic neuropathy. Diabetes 2004; 53(7):1824-1830.
(7) Auer R N. Automated nerve fibre size and myelin sheath measurement using microcomputer-based digital image analysis: theory, method and results. J Neurosci Methods 1994; 51:229-238.
(8) Singhal A, Cheng C, Sun H, Zochodne D W. Near nerve local insulin prevents conduction slowing in experimental diabetes. Brain Res 1997; 763(2):209-214.
(9) O'Brien P C, Shampo M A. Statistical considerations for performing multiple tests in a single experiment. Mayo Clin Proc 1988; 63:813-820.
(10) Zochodne D W. Nerve and ganglion blood flow in diabetes: an appraisal. In: Tomlinson D, ed. Neurobiology of diabetic neuropathy. San Diego: Academic Press, 2002:161-202.
(11) Zochodne D W, Ho L T. The influence of sulindac on experimental streptozotocin-induced diabetic neuropathy. Can J Neurol Sci 1994; 21(3): 194-202.
(12) Sima A A F, Brismar T, Yagihashi S, Neuropathies encountered in the spontaneously diabetic BB Wistar rat. In: Dyck P J, Thomas P K, Asbury A K, Winegrad A I, Porte D, Jr., eds. Diabetic Neuropathy. Toronto: W.B. Saunders, 1987.

### Other Embodiments

From the foregoing description, it will be apparent to one of ordinary skill in the art that variations and modifications may be made to the invention described herein to adapt it to various usages and conditions. Such embodiments are also within the scope of the present invention.

All publications mentioned in this specification are hereby incorporated by reference.

1-53. (canceled)

54. A method of treating and/or prophylaxis of neuropathic pain comprising: administering to a subject suffering from neuropathic pain, a therapeutically effective amount of a compound, according to Formula Ia: 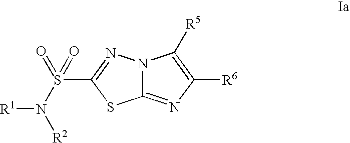
or a salt thereof,
wherein:
n is 1 or 2;
Y is NH, O or S;
R¹ and R² are independently selected from:
1) H,
2) C₁-C₆ alkyl,
R⁵ is:
1) H,
2) halogen,
3) C₁-C₆ alkyl,
wherein the aryl and the heteroaryl are optionally substituted with one or more R²⁰ substituents;
R⁶ is
1) adamantyl,
2) aryl,
3) heteroaryl,
4) fused phenyl-cycloalkyl substituted with alkyl, or
5) fused phenyl-heterocyclyl optionally substituted with cycloalkyl,
wherein the aryl and the heteroaryl are optionally substituted with one or more substituents independently selected from R²⁰;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, heterocyclyl, and biphenyl are optionally substituted with one or more R²⁰ substituents;
R¹¹ and R¹² are independently selected from:
1) C₁-C₆ alkyl,
2) C₃-C₇ cycloalkyl,
3) haloalkyl,
4) aryl,
5) heteroaryl,
6) heterocyclyl,
7) CO—C₁-C₆ alkyl
8) CO—C₃-C₇ cycloalkyl
9) CO-aryl,
10) CO-heteroaryl,
11) CO-heterocyclyl,
12) C(O)Y—C₁-C₆ alkyl
13) C(O)Y—C₃-C₇ cycloalkyl
14) C(O)Y-aryl,
15) C(O)Y-heteroaryl, or
16) C(O)Y-heterocyclyl,
wherein the alkyl and the cycloalkyl are optionally substituted with one or more R¹⁵ substituents, and the aryl, heteroaryl, heterocyclyl, and biphenyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁵ is
1) NO₂,
2) CN,
3) halogen,
4) C₁-C₆ alkyl,
5) C₃-C₇ cycloalkyl,
6) haloalkyl,
7) aryl,
8) heteroaryl,
9) heterocyclyl,
10) OR¹⁰,
11) S(O)ₙR¹⁰,
12) NR¹¹R¹²,
13) COR¹⁰,
14) CO₂R¹⁴,
15) CONR¹¹R¹², or
16) S(O)ₙNR¹¹R¹²,
wherein the aryl and heteroaryl are optionally substituted with one or more R¹⁰ substituents;
R²⁰ is
1) NO₂,
2) CN,
3) N₃,
4) B(OH)₂,
5) adamantyl,
6) halogen,
7) C₁-C₆ alkyl,
8) C₃-C₇ cycloalkyl,
9) aryl,
10) heteroaryl,
11) heterocyclyl,
12) fused phenyl heterocyclyl,
13) haloalkyl,
14) OR¹⁰,
15) SR¹⁰,
16) S(O)ₙR¹⁰,
17) NR¹¹R¹², or
18) COR¹⁰,
wherein the alkyl, the aryl, the heteroaryl, the heterocyclyl, and the cycloalkyl are optionally substituted with one or more R¹⁵ substituents.

55. The method according to claim 54, in which the compound is a pharmaceutically acceptable salt.

56. The method according to claim 54, in which R¹ and R² are individually selected from the group consisting of H, methyl, ethyl, propyl, and butyl.

57. The method according to claim 56, in which R¹ and R² are both H.

58. The method according to claim 54, in which R⁵ is H.

59. The method according to claim 54, in which R⁶ is
1) adamantyl,
2) aryl,
3) heteroaryl,
4) fused phenyl-cycloalkyl substituted with alkyl, or
5) fused phenyl-heterocyclyl optionally substituted with cycloalkyl
wherein the aryl and the heteroaryl are optionally substituted with one or more substituents independently selected from R²⁰.

60. The method according to claim 59, in which R⁶ is phenyl optionally substituted with one or more R²⁰ substituents.

61. The method according to claim 60, in which R⁶ is selected from the group consisting of:

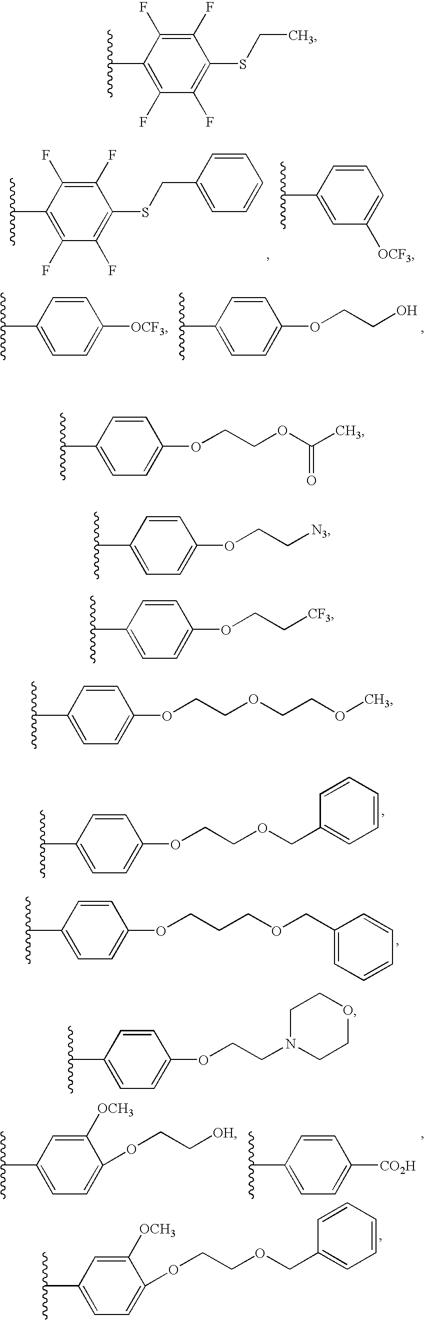

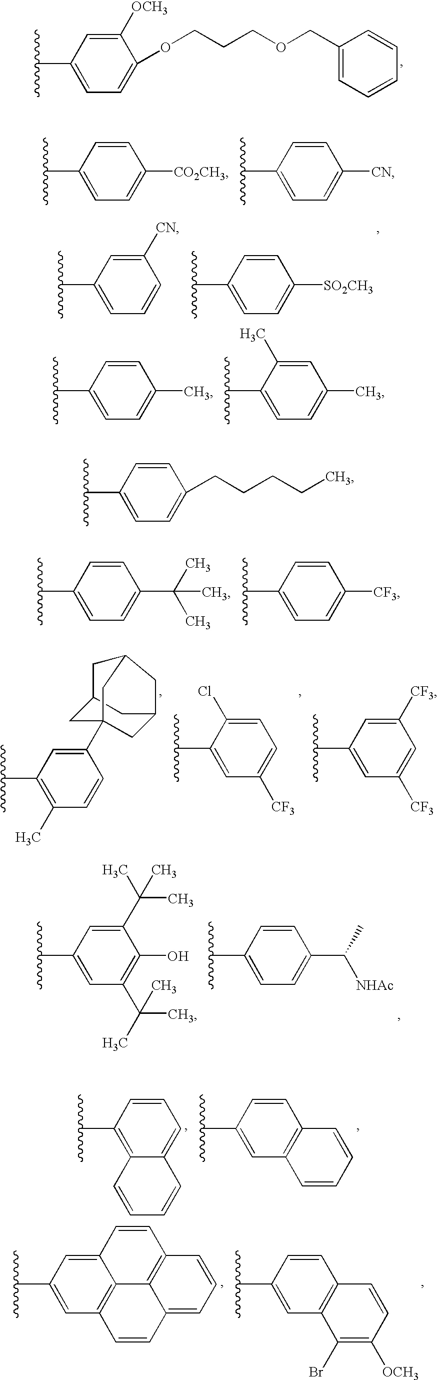

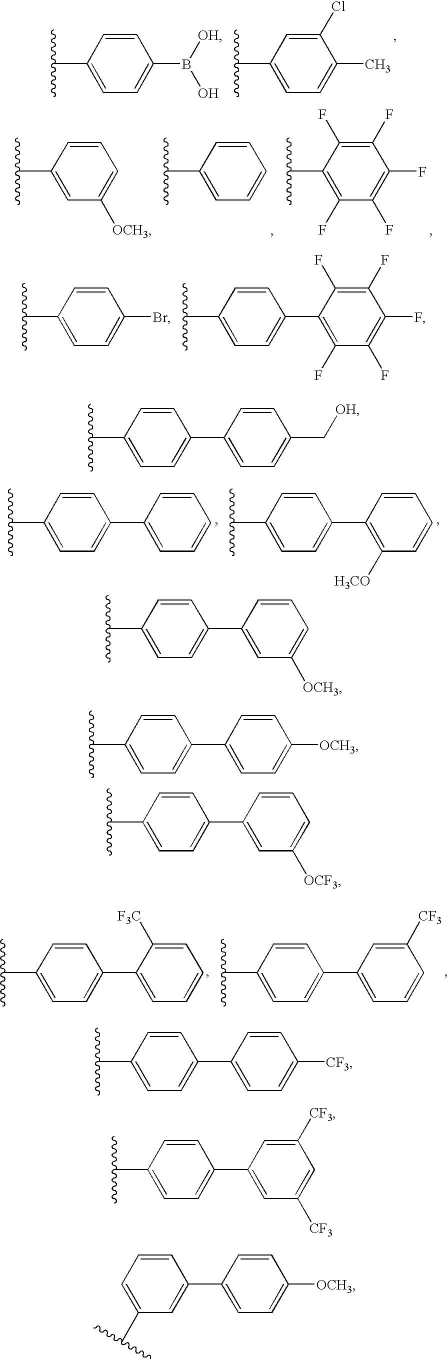

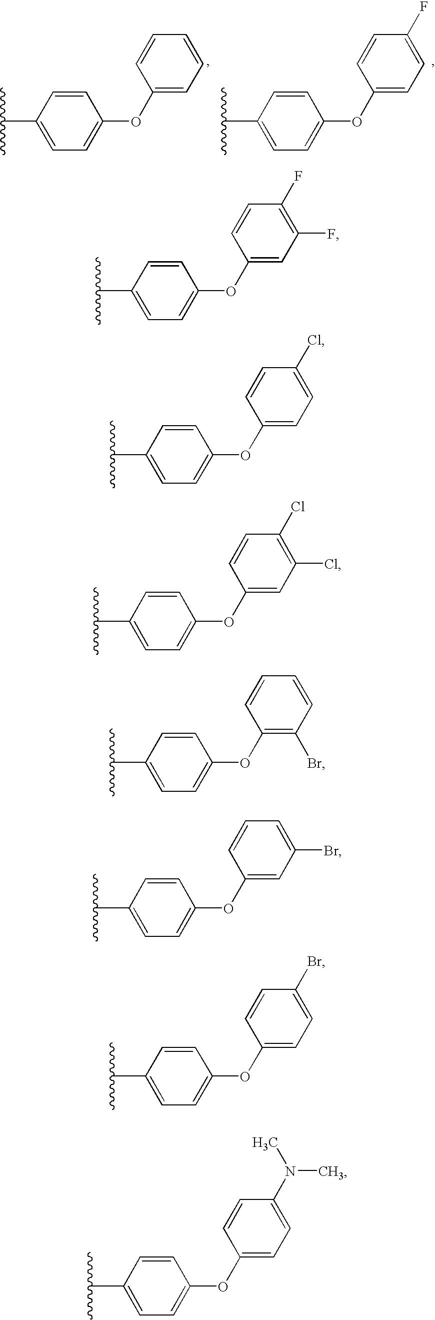

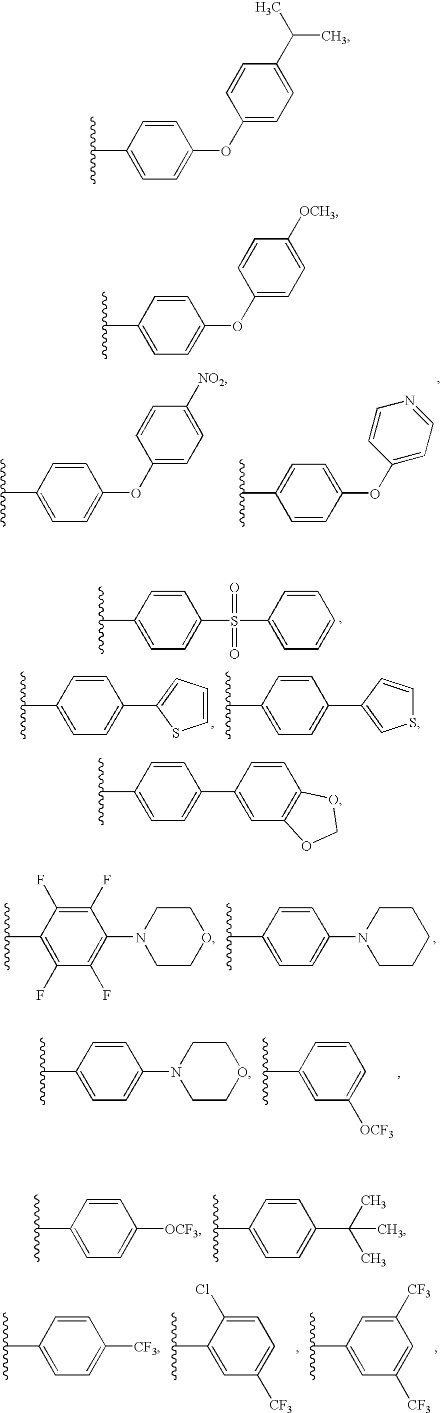

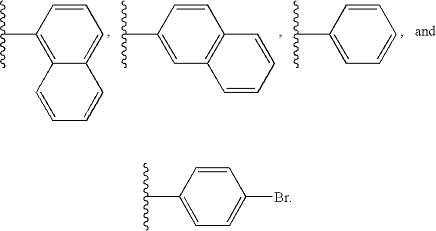

62. The method according to claim 61, in which R⁶ is heteroaryl, fused phenyl-cycloalkyl substituted with two or more methyl groups, or fused phenyl-heterocyclyl substituted with cyclohexane.

63. The method according to claim 62, in which R⁶ is selected from the group consisting of:

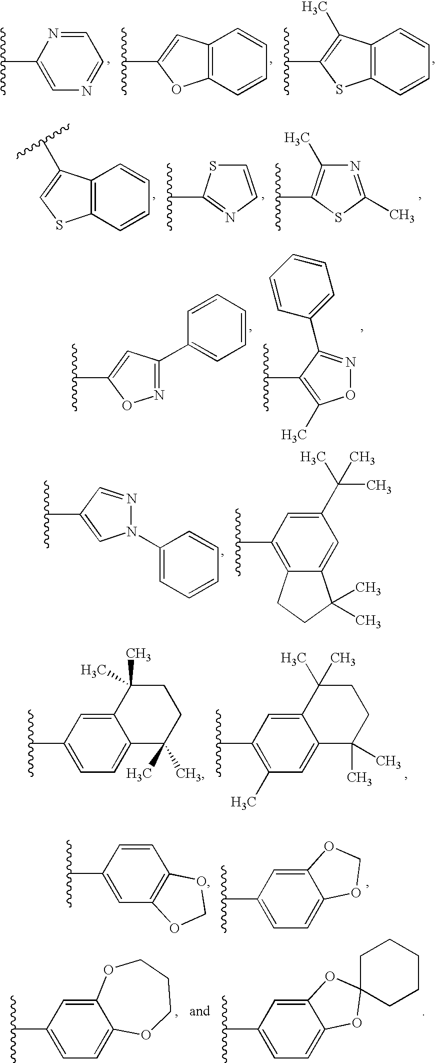

64. The method according to claim 54, in which the compound is selected from the group consisting of: compound nos. 12, 154, 21, 155, 24, 156, 30, 157, 49, 158, 52, 159, 53, 160, 81, and 150.

65. The method according to claim 54, in which the compound is administered subcutaneously, intramuscularly, intravenously or orally.

66. The method according to claim 54, in which the subject is a human.

67. The method according to claim 54, in which the neuropathic pain is caused by peripheral nerve trauma, entrapment neuropathy, nerve transaction, including surgery, causaglia, amputation and stump pain, neuroma, and post-choracotomy pain, mononeuropathies such as diabetic, malignant nerve/plexus invasion, ischemic irradiation, connective tissue disease, polyneuropathies such as diabetic, alcoholic, nutritional, amyloid, Fabry disease, chemical (e.g., chemotherapeutic agents), idiopathic and AIDS neuropathy; root and dorsal root ganglion, prolapsed disk/compression, postherpetic or trigeminal neuralgia, arachnoiditis, root avulsion, tumor compression and surgical rhizotomy; by spinal cord injury such as trauma, transaction, hemisection, Lissauer tract section, syrinx, multiple sclerosis, tumor compression, arteriovenous malformation, Dyscraphism, Vitamin B12 deficiency, hematomyelia, syphilitic myelitis, and Commissural myelotomy; brain stem injury such as Wallenberg's syndrome, tuberculoma, tumor, and syrinx; thalamus injury, such as infarction, tumor, surgical lesions in main, sensory nucleus, and hemorrahage; corrical/subcorrical injury, such as infarction, trauma, tumor, and arteriovenous malformation, painful diabetic peripheral neuropathy, post-herpetic neuralgia, trigeminal neuralgia, post-stroke pain, multiple sclerosis-associated pain, neuropathies-associated pain such as in idiopathic or post-traumatic neuropathy and mononeuritis, HIV-associated neuropathic pain, cancer-associated neuropathic pain, carpal tunnel-associated neuropathic pain, spinal cord injury-associated pain, complex regional pain syndrome, fibromyalgia-associated neuropathic pain, lumbar and cervical pain, reflex sympathic dystrophy, phantom limb syndrome and other chronic and debilitating condition-associated pain syndromes.

68. The method according to claim 67, in which the neuropathic pain is caused by diabetic neuropathy.

69. The method according to claim 54, in which the compound of Formula Ia reduces tactile allodynia.

70. A pharmaceutical composition for treating and/or prophylaxis of neuropathic pain, comprising: a pharmaceutically acceptable carrier and a therapeutically effective amount of a compound, according to Formula Ia 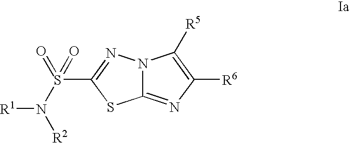
or a salt thereof,
wherein:
n is 1 or 2;
Y is NH, O or S;
R¹ and R² are independently selected from:
1) H,
2) C₁-C₆ alkyl,
R⁵ is:
1) H,
2) halogen,
3) C₁-C₆ alkyl,
wherein the aryl and the heteroaryl are optionally substituted with one or more R²⁰ substituents;
R⁶ is
1) adamantyl,
2) aryl,
3) heteroaryl,
4) fused phenyl-cycloalkyl substituted with alkyl, or
5) fused phenyl-heterocyclyl optionally substituted with cycloalkyl,
wherein the aryl and the heteroaryl are optionally substituted with one or more substituents independently selected from R²⁰;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, heterocyclyl, and biphenyl are optionally substituted with one or more R²⁰ substituents;
R¹¹ and R¹² are independently selected from:
1) C₁-C₆ alkyl,
2) C₃-C₇ cycloalkyl,
3) haloalkyl,
4) aryl,
5) heteroaryl,
6) heterocyclyl,
7) CO—C₁-C₆ alkyl
8) CO—C₃-C₇ cycloalkyl
9) CO-aryl,
10) CO-heteroaryl,
11) CO-heterocyclyl,
12) C(O)Y—C₁-C₆ alkyl
13) C(O)Y—C₃-C₇ cycloalkyl
14) C(O)Y-aryl,
15) C(O)Y-heteroaryl, or
16) C(O)Y-heterocyclyl,
wherein the alkyl and the cycloalkyl are optionally substituted with one or more R¹⁵ substituents, and the aryl, heteroaryl, heterocyclyl, and biphenyl are optionally substituted with one or more R²⁰ substituents;
or R¹″ and R¹² together with the nitrogen atom to which they are bonded form a five, six or seven membered heterocyclic ring optionally substituted with one or more R²⁰ substituents;
R¹⁵ is
1) NO₂,
2) CN,
3) halogen,
4) C₁-C₆ alkyl,
5) C₃-C₇ cycloalkyl,
6) haloalkyl,
7) aryl,
8) heteroaryl,
9) heterocyclyl,
10) OR¹⁰,
11) S(O)ₙR¹⁰,
12) NR¹¹R¹²,
13) COR¹⁰,
14) CO₂R¹⁴,
15) CONR¹¹R¹², or
16) S(O)ₙNR¹¹R¹²,
or R¹¹ and R¹² together with the nitrogen atom to which they are bonded form a five, six or seven membered heterocyclic ring optionally substituted with one or more R²⁰ substituents;
R¹⁵ is
1) NO₂,
2) CN,
3) halogen,
4) C₁-C₆ alkyl,
5) C₃-C₇ cycloalkyl,
6) haloalkyl,
7) aryl,
8) heteroaryl,
9) heterocyclyl,
10) OR¹⁰,
11) S(O)ₙR¹⁰,
12) NR¹¹R¹²,
13) COR¹⁰,
14) CO₂R¹⁴,
15) CONR¹¹R¹², or
16) S(O)ₙNR¹¹R¹²,
wherein the aryl and heteroaryl are optionally substituted with one or more R¹⁰ substituents;
R²⁰ is
1) NO₂,
2) CN,
3) N₃,
4) B(OH)₂,
5) adamantyl,
6) halogen,
7) C₁-C₆ alkyl,
8) C₃-C₇ cycloalkyl,
9) aryl,
10) heteroaryl,
11) heterocyclyl,
wherein the aryl and heteroaryl are optionally substituted with one or more R¹⁰ substituents;
R²⁰ is
1) NO₂,
2) CN,
3) N₃,
4) B(OH)₂,
5) adamantyl,
6) halogen,
7) C₁-C₆ alkyl,
8) C₃-C₇ cycloalkyl,
9) aryl,
10) heteroaryl,
11) heterocyclyl,
12) fused phenyl heterocyclyl,
13) haloalkyl,
14) OR¹⁰,
15) SR¹⁰,
16) S(O)ₙR¹⁰,
17) NR¹¹R¹², or
18) COR¹⁰,
wherein the alkyl, the aryl, the heteroaryl, the heterocyclyl, and the cycloalkyl are optionally substituted with one or more R¹⁵ substituents.

71. Use of a compound of Formula Ia 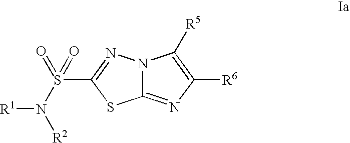
or a salt thereof,
wherein:
n is 1 or 2;
Y is NH, O or S;
R¹ and R² are independently selected from:
1) H,
2) C₁-C₆ alkyl,
R⁵ is:
1) H,
2) halogen,
3) C₁-C₆ alkyl,
wherein the aryl and the heteroaryl are optionally substituted with one or more R²⁰ substituents;
R⁶ is
1) adamantyl,
2) aryl,
3) heteroaryl,
4) fused phenyl-cycloalkyl substituted with alkyl, or
5) fused phenyl-heterocyclyl optionally substituted with cycloalkyl,
wherein the aryl and the heteroaryl are optionally substituted with one or more substituents independently selected from R²⁰;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, heterocyclyl, and biphenyl are optionally substituted with one or more R²⁰ substituents;
R¹¹ and R¹² are independently selected from:
1) C₁-C₆ alkyl,
2) C₃-C₇ cycloalkyl,
3) haloalkyl,
4) aryl,
5) heteroaryl,
6) heterocyclyl,
7) CO—C₁-C₆ alkyl
8) CO—C₃-C₇ cycloalkyl
9) CO-aryl,
10) CO-heteroaryl,
11) CO-heterocyclyl,
12) C(O)Y—C₁-C₆ alkyl
13) C(O)Y—C₃-C₇ cycloalkyl
14) C(O)Y-aryl,
15) C(O)Y-heteroaryl, or
16) C(O)Y-heterocyclyl,
wherein the alkyl and the cycloalkyl are optionally substituted with one or more R¹⁵ substituents, and the aryl, heteroaryl, heterocyclyl, and biphenyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁵ is
1) NO₂,
2) CN,
3) halogen,
4) C₁-C₆ alkyl,
5) C₃-C₇ cycloalkyl,
6) haloalkyl,
7) aryl,
8) heteroaryl,
9) heterocyclyl,
10) OR¹⁰,
11) S(O)ₙR¹⁰,
12) NR¹¹R¹²,
13) COR¹⁰,
14) CO₂R¹⁴,
15) CONR¹¹R¹², or
16) S(O)ₙNR¹¹R¹²,
wherein the aryl and heteroaryl are optionally substituted with one or more R¹⁰ substituents;
R²⁰ is
1) NO₂,
2) CN,
3) N₃,
4) B(OH)₂,
5) adamantyl,
6) halogen,
7) C₁-C₆ alkyl,
8) C₃-C₇ cycloalkyl,
9) aryl,
10) heteroaryl,
11) heterocyclyl,
12) fused phenyl heterocyclyl,
13) haloalkyl,
14) OR¹⁰,
15) SR¹⁰,
16) S(O)ₙR¹⁰,
17) NR¹¹R¹² or
18) COR¹⁰,
wherein the alkyl, the aryl, the heteroaryl, the heterocyclyl, and the cycloalkyl are optionally substituted with one or more R¹⁵ substituents, for the treatment and/or prophylaxis of neuropathic pain in a subject.

72. Use of a compound of Formula Ia 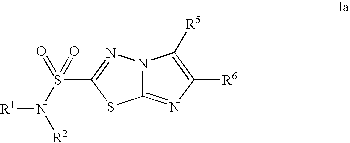
or a salt thereof,
wherein:
n is 1 or 2;
Y is NH, O or S;
R¹ and R² are independently selected from:
1) H,
2) C₁-C₆ alkyl,
R⁵ is:
1) H,
2) halogen,
3) C₁-C₆ alkyl,
wherein the aryl and the heteroaryl are optionally substituted with one or more R²⁰ substituents;
R⁶ is
1) adamantyl,
2) aryl,
3) heteroaryl,
4) fused phenyl-cycloalkyl substituted with alkyl, or
5) fused phenyl-heterocyclyl optionally substituted with cycloalkyl,
wherein the aryl and the heteroaryl are optionally substituted with one or more substituents independently selected from R²⁰;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, heterocyclyl, and biphenyl are optionally substituted with one or more R²⁰ substituents;
R¹¹ and R¹² are independently selected from:
1) C₁-C₆ alkyl,
2) C₃-C₇ cycloalkyl,
3) haloalkyl,
4) aryl,
5) heteroaryl,
6) heterocyclyl,
7) CO—C₁-C₆ alkyl
8) CO—C₃-C₇ cycloalkyl
9) CO-aryl,
10) CO-heteroaryl,
11) CO-heterocyclyl,
12) C(O)Y—C₁-C₆ alkyl
13) C(O)Y—C₃-C₇ cycloalkyl
14) C(O)Y-aryl,
15) C(O)Y-heteroaryl, or
16) C(O)Y-heterocyclyl,
wherein the alkyl and the cycloalkyl are optionally substituted with one or more R¹⁵ substituents, and the aryl, heteroaryl, heterocyclyl, and biphenyl are optionally substituted with one or more R²⁰ substituents;
12) fused phenyl heterocyclyl,
13) haloalkyl,
14) OR¹⁰,
15) SR¹⁰,
16) S(O)ₙR¹⁰,
17) NR¹¹R¹², or
18) COR¹⁰,
wherein the alkyl, the aryl, the heteroaryl, the heterocyclyl, and the cycloalkyl are optionally substituted with one or more R¹⁵ substituents,
in the manufacture of a medicament for the treatment and/or prophylaxis of neuropathic pain in a subject.

73. The use according to claim 71, in which the compound is a pharmaceutically acceptable salt.

74. The use according to claim 71, in which R¹ and R² are individually selected from the group consisting of H, methyl, ethyl, propyl, and butyl.

75. The use according to claim 74, in which R¹ and R² are both H.

76. The use according to claim 71, in which R⁵ is H.

77. The use according to claim 71, in which R⁵ is
1) aryl,
2) heteroaryl,
3) fused phenyl-cycloalkyl substituted with alkyl, or
4) fused phenyl-heterocyclyl optionally substituted with cycloalkyl
wherein the aryl and the heteroaryl are optionally substituted with one or more substituents independently selected from R²⁰.

78. The use according to claim 77, in which R⁶ is phenyl optionally substituted with one or more R²⁰ substituents.

79. The use, according to claim 78, in which R⁵ is selected from the group consisting of:

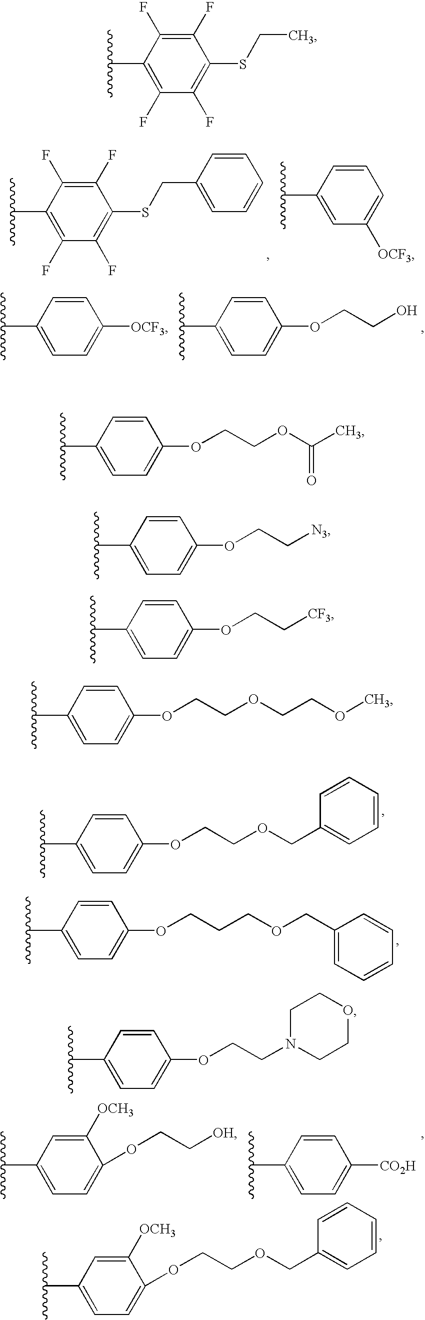

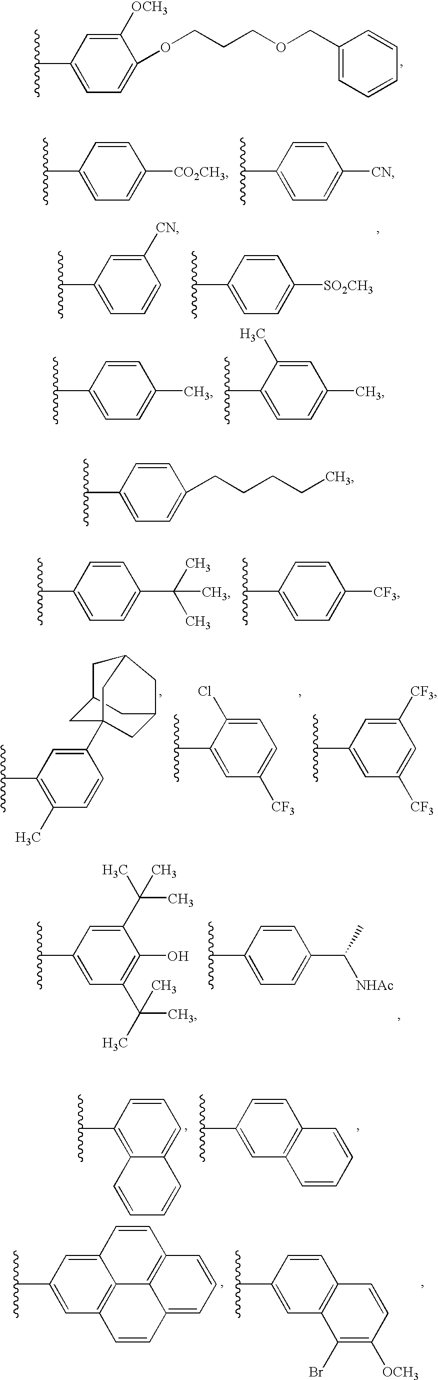

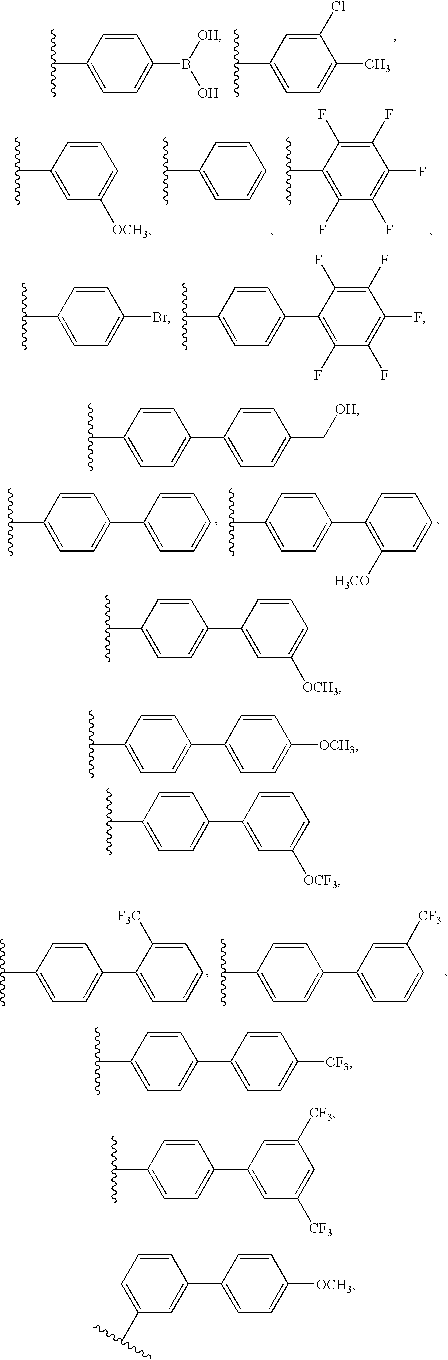

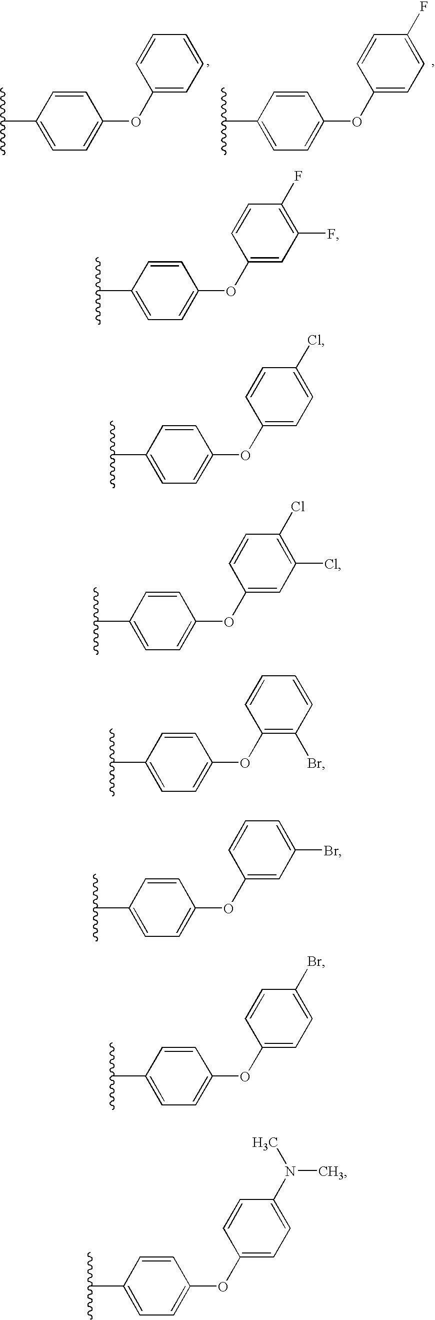

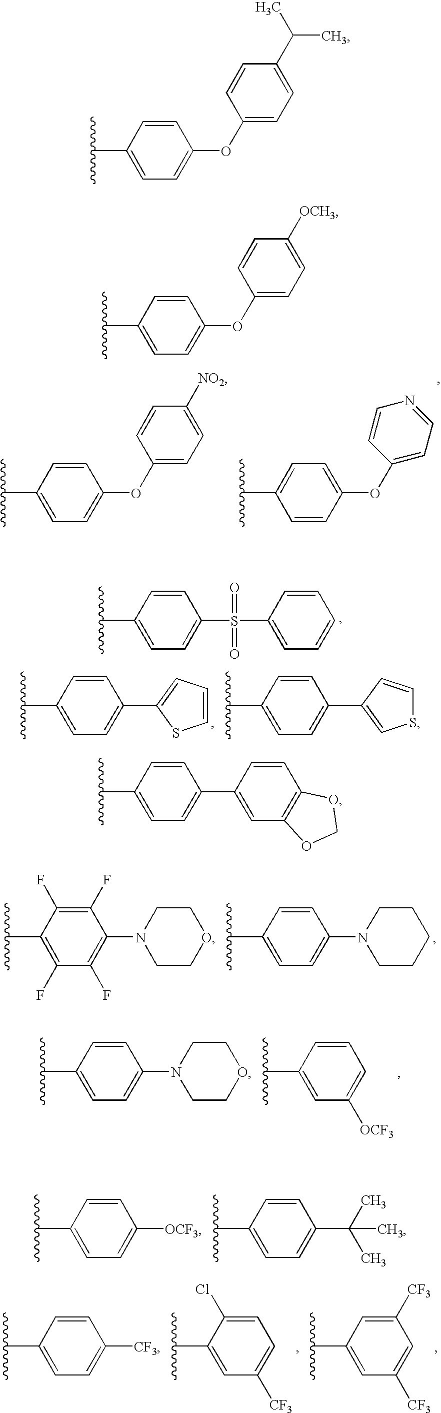

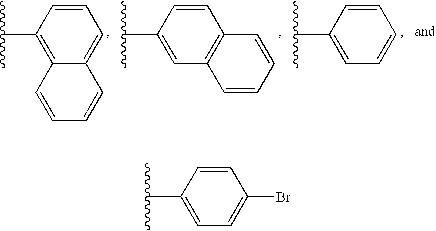

80. The use according to claim 77, in which R⁶ is heteroaryl, fused phenyl-cycloalkyl substituted with two or more methyl groups, or fused phenyl-heterocyclyl substituted with cyclohexane.

81. The use according to claim 80, in which R⁶ is selected from the group consisting of:

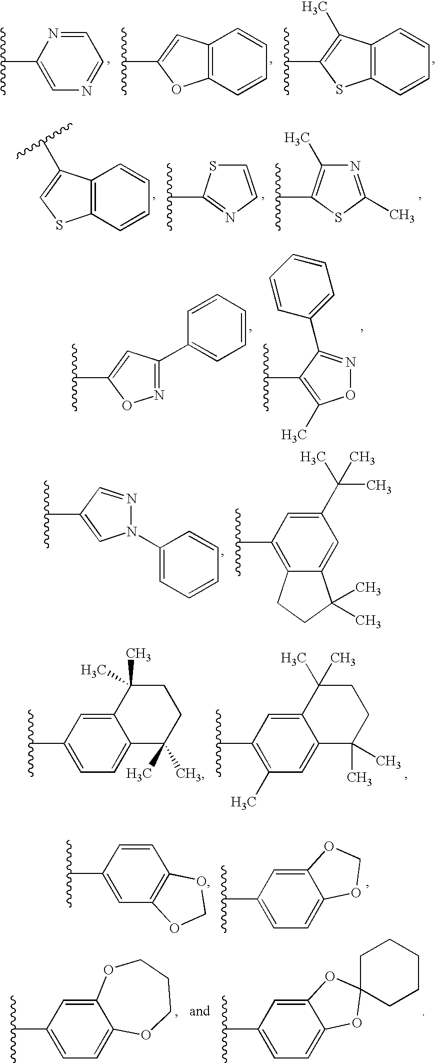

82. The use, according to claim 71, in which the compound is selected from the group consisting of: compound nos. 12, 154, 21, 155, 24, 156, 30, 157, 49, 158, 52, 159, 53, 160, 81 and 150.

83. The use according to claim 71, in which the compound is administered subcutaneously, intramuscularly, intravenously or orally.

84. The use according to claim 71, in which the subject is a human.

85. The method according to claim 71, in which the neuropathic pain is caused by peripheral nerve trauma, entrapment neuropathy, nerve transaction, including surgery, causaglia, amputation and stump pain, neuroma, and post-choracotomy pain, mononeuropathies such as diabetic, malignant nerve/plexus invasion, ischemic irradiation, connective tissue disease, polyneuropathies such as diabetic, alcoholic, nutritional, amyloid, Fabry disease, chemical (e.g., chemotherapeutic agents), idiopathic and AIDS neuropathy; root and dorsal root ganglion, prolapsed disk/compression, postherpetic or trigeminal neuralgia, arachnoiditis, root avulsion, tumor compression and surgical rhizotomy; by spinal cord injury such as trauma, transaction, hemisection, Lissauer tract section, syrinx, multiple sclerosis, tumor compression, arteriovenous malformation, Dyscraphism, Vitamin B12 deficiency, hematomyelia, syphilitic myelitis, and Commissural myelotomy; brain stem injury such as Wallenberg's syndrome, tuberculoma, tumor, and syrinx; thalamus injury, such as infarction, tumor, surgical lesions in main, sensory nucleus, and hemorrahage; corrical/subcorrical injury, such as infarction, trauma, tumor, and arteriovenous malformation, painful diabetic peripheral neuropathy, post-herpetic neuralgia, trigeminal neuralgia, post-stroke pain, multiple sclerosis-associated pain, neuropathies-associated pain such as in idiopathic or post-traumatic neuropathy and mononeuritis, HIV-associated neuropathic pain, cancer-associated neuropathic pain, carpal tunnel-associated neuropathic pain, spinal cord injury-associated pain, complex regional pain syndrome, fibromyalgia-associated neuropathic pain, lumbar and cervical pain, reflex sympathic dystrophy, phantom limb syndrome and other chronic and debilitating condition-associated pain syndromes.

86. The use according to claim 85, in which the neuropathic pain is caused by diabetic neuropathy.

87. The use according to claim 71, in which the compound of Formula I reduces tactile allodynia.
